(19) United States
(12) Patent Application Publication
(10) Pub. No.: US 2024/0220685 A1
(43) Pub. Date: Jul. 4, 2024

(54) METHOD FOR TRAINING BINDING AFFINITY DETECTION MODEL AND BINDING AFFINITY DETECTION METHOD

(71) Applicant: Tencent Technology (Shenzhen) Company Limited, Shenzhen (CN)

(72) Inventor: Shaoyong XU, Shenzhen (CN)

(73) Assignee: Tencent Technology (Shenzhen) Company Limited, Sehnzhen (CN)

(21) Appl. No.: 18/434,081

(22) Filed: Feb. 6, 2024

Related U.S. Application Data
(63) Continuation of application No. PCT/CN2023/113188, filed on Aug. 15, 2023.

(30) Foreign Application Priority Data
Sep. 23, 2022 (CN) 2022111682437

(51) Int. Cl. G06F 30/27

(52) U.S. Cl. CPC G06F 30/27

(57) ABSTRACT

A method/apparatus for training a binding affinity detection model including obtaining protein structure data of a sample protein, small molecule structure data of a sample small drug molecule, and a sample labeling result, calling a neural network model to determine structure data of a plurality of target complex conformations based on the protein structure data and the small molecule structure data, calling the neural network model to determine a sample prediction result based on the structure data of the target complex conformations, and training the neural network model using the sample prediction result and the sample labeling result to obtain a binding affinity detection model to detect a binding affinity between a target protein and a target small drug molecule.

### CROSS-REFERENCE TO RELATED APPLICATIONS

This application is a continuation application of International Application No. PCT/CN2023/113188 filed on Aug. 15, 2023, which claims priority to Chinese Patent Application No. 202211168243.7 filed with the China National Intellectual Property Administration on Sep. 23, 2022, the disclosures of each being incorporated by reference herein in their entireties.

### FIELD

The disclosure relates to the field of biotechnologies, and specifically, to a method for training a binding affinity detection model and a binding affinity detection method.

### BACKGROUND

In the field of biotechnologies, proteins participate in life activities such as catalysis, immunity, and metabolism, and are important substances that form organisms. Generally, a binding affinity between a protein and a small drug molecule may be measured, and by analyzing the binding affinity, a druggability potential of the small drug molecule may be obtained.

In the related art, a binding affinity detection model may be used to detect the binding affinity between the protein and the small drug molecule. Since the binding affinity between the protein and the small drug molecule is closely related to the druggability potential of the small drug molecule, how to train a binding affinity detection model with higher accuracy become important.

### SUMMARY

Some embodiments provide a method for training a binding affinity detection model and a binding affinity detection method, which may be configured for improving accuracy of the binding affinity detection model. The technical solutions include the following content.

Some embodiments provide a method for training a binding affinity detection model is provided, performed by an electronic device, and the method including: obtaining protein structure data of a sample protein, small molecule structure data of a sample small drug molecule, and a sample labeling result, the sample labeling result being obtained through labeling and indicating a binding affinity between the sample protein and the sample small drug molecule; calling a neural network model to determine structure data of a plurality of target complex conformations based on the protein structure data and the small molecule structure data; calling the neural network model to determine a sample prediction result based on the structure data of the target complex conformations; and training the neural network model using the sample prediction result and the sample labeling result to obtain a binding affinity detection model to detect a binding affinity between a target protein and a target small drug molecule.

Some embodiments provide an apparatus for training a binding affinity detection model is provided, including: at least one memory configured to store program code; and at least one processor configured to read the program code and operate as instructed by the program code, the program code comprising: obtaining code configured to cause at least one of the at least one processor to obtain protein structure data of a sample protein, small molecule structure data of a sample small drug molecule, and a sample labeling result, the sample labeling result being obtained through labeling and indicating a binding affinity between the sample protein and the sample small drug molecule; determining code configured to cause at least one of the at least one processor to: call a neural network model to determine structure data of a plurality of target complex conformations based on the protein structure data and the small molecule structure data; call the neural network model to determine a sample prediction result based on the structure data of the target complex conformations; and training code configured to cause at least one of the at least one processor to train the neural network model using the sample prediction result and the sample labeling result to obtain a binding affinity detection model to detect a binding affinity between a target protein and a target small drug molecule.

Some embodiments provide a non-transitory computer-readable storage medium storing computer code which, when executed by at least one processor, causes the at least one processor to at least: obtain protein structure data of a sample protein, small molecule structure data of a sample small drug molecule, and a sample labeling result, the sample labeling result being obtained through labeling and indicating a binding affinity between the sample protein and the sample small drug molecule; call a neural network model to determine structure data of a plurality of target complex conformations based on the protein structure data and the small molecule structure data; call the neural network model to determine a sample prediction result based on the structure data of the target complex conformations; and train the neural network model using the sample prediction result and the sample labeling result to obtain a binding affinity detection model configured to detect a binding affinity between a target protein and a target small drug molecule.

In some embodiments, any target complex conformation is a structure obtained through prediction and formed by binding of a sample protein and a sample small drug molecule. Through a plurality of target complex conformations, a probability of covering a real structure formed by the binding of the sample protein and the sample small drug molecule may be improved. That is, the plurality of target complex conformations can more accurately express the real structure formed by the binding of the sample protein and the sample small drug molecule, so that a sample prediction result is more accurate when a neural network model is called to determine the sample prediction result based on structure data of the target complex conformations. In this way, when the neural network model is trained by using the sample prediction result to obtain a binding affinity detection model, the accuracy of the binding affinity detection model can be improved, thereby improving the accuracy of a binding affinity detection result.

### BRIEF DESCRIPTION OF THE DRAWINGS

To describe the technical solutions of some embodiments of this disclosure more clearly, the following briefly introduces the accompanying drawings for describing some embodiments. The accompanying drawings in the following description show only some embodiments of the disclosure, and a person of ordinary skill in the art may still derive other drawings from these accompanying drawings without creative efforts. In addition, one of ordinary skill would understand that aspects of some embodiments may be combined together or implemented alone.

FIG. 1 is a schematic diagram of an implementation environment of a method for training a binding affinity detection model or a binding affinity detection method according to some embodiments.

FIG. 2 is a flowchart of a method for training a binding affinity detection model according to some embodiments.

FIG. 3 is a schematic diagram of a spatial structure and a graph structure of a candidate complex conformation according to some embodiments.

FIG. 4 is a schematic diagram of extracting a conformational feature of a candidate complex conformation according to some embodiments.

FIG. 5 is a schematic diagram of determining a weighted fusion feature according to some embodiments.

FIG. 6 is a schematic diagram of determining a sample prediction result according to some embodiments.

FIG. 7 is a schematic diagram of training a binding affinity detection model according to some embodiments.

FIG. 8 is a flowchart of a binding affinity detection method according to some embodiments.

FIG. 9 is a schematic diagram of a binding affinity detection process according to some embodiments.

FIG. 10 is a schematic diagram of comparison of test indicators according to some embodiments.

FIG. 11 is a schematic structural diagram of an apparatus for training a binding affinity detection model according to some embodiments.

FIG. 12 is a schematic structural diagram of a binding affinity detection apparatus according to some embodiments.

FIG. 13 is a schematic structural diagram of a terminal device according to some embodiments.

FIG. 14 is a schematic structural diagram of a server according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure and the appended claims.

In the following descriptions, related “some embodiments” describe a subset of all possible embodiments. However, it may be understood that the “some embodiments” may be the same subset or different subsets of all the possible embodiments, and may be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

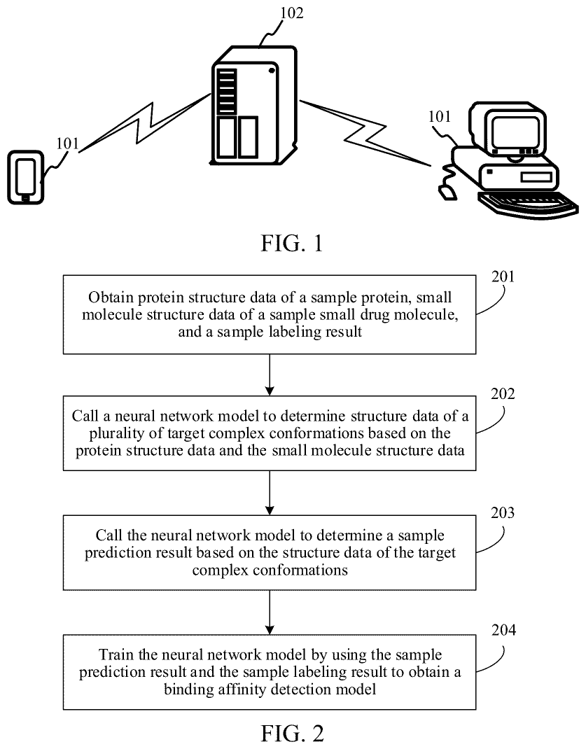

FIG. 1 is a schematic diagram of an implementation environment of a method for training a binding affinity detection model or a binding affinity detection method according to some embodiments. As shown in FIG. 1, the implementation environment includes a terminal device 101 and a server 102. The method for training a binding affinity detection model or the binding affinity detection method in some embodiments may be performed by the terminal device 101, or may be performed by the server 102, or may be performed by the terminal device 101 and the server 102 together.

The terminal device 101 may be a smartphone, a game console, a desktop computer, a tablet computer, a laptop portable computer, a smart television, a smart in-vehicle device, a smart speech interaction device, or a smart household appliance. The server 102 may be one server, a server cluster including a plurality of servers, or any one of a cloud computing platform and a virtualization center. This is not limited herein. The server 102 is communicatively connected to the terminal device 101 through a wired network or a wireless network. The server 102 may have functions such as data processing, data storage, and data sending and receiving. This is not limited herein. A quantity of the terminal devices 101 and a quantity of the servers 102 are not limited, and there may be one or more terminal devices 101 and servers 102.

The method for training a binding affinity detection model and the binding affinity detection method provided in some embodiments are both implemented based on an artificial intelligence (AI) technology. The AI technology is a theory, method, technology and application system that uses a digital computer or a machine controlled by the digital computer to simulate, extend and expand human intelligence, perceive an environment, acquire knowledge, and use the knowledge to obtain an optimal result. In other words, AI is a comprehensive technology in computer science and attempts to understand the essence of intelligence and produce a new intelligent machine that can react in a manner similar to human intelligence. The AI is to study design principles and implementation methods of various intelligent machines, to enable a machine to have functions of perception, reasoning, and decision-making.

The AI technology is a comprehensive discipline, relating to a wide range of fields, and involving both a hardware-level technology and a software-level technology. Basic AI technologies generally include technologies such as a sensor, a dedicated AI chip, cloud computing, distributed storage, a big data processing technology, an operating/interaction system, and electromechanical integration. AI software technologies mainly include fields such as a computer vision technology, a speech processing technology, a natural language processing technology, machine learning/deep learning, automatic driving, and intelligent transportation.

In the field of biotechnologies, by analyzing a binding affinity between a protein and a small drug molecule, a druggability potential of the small drug molecule may be analyzed. Therefore, detecting the binding affinity between the protein and the small drug molecule is a crucial technology.

Generally, a binding affinity detection model may be trained by using structure data of a sample protein and structure data of a sample small drug molecule, and a binding affinity between a target protein and a target small drug molecule is detected by using the binding affinity detection model. Since the binding affinity between the protein and the small drug molecule is closely related to the druggability potential of the small drug molecule, how to train a binding affinity detection model with higher accuracy becomes an important technology.

Some embodiments provide a method for training a binding affinity detection model. The method may be applied in the foregoing implementation environment and may improve the accuracy of the binding affinity detection model. A flowchart of a method for training a binding affinity detection model according to some embodiments shown in FIG. 2 is used as an example. For ease of description, the terminal device 101 or the server 102 that performs the method for training a binding affinity detection model in some embodiments is referred to as an electronic device, and the method may be performed by the electronic device. As shown in FIG. 2, the method includes the following operations:

Operation 201: Obtain protein structure data of a sample protein, small molecule structure data of a sample small drug molecule, and a sample labeling result.

The protein is a substance with a certain spatial structure (such as a three-dimensional structure) formed by twisting and folding of a polypeptide chain. The polypeptide chain is a substance formed by amino acids through dehydration and condensation. Since the protein has a certain spatial structure, the protein structure data of the sample protein may be used to represent a spatial structure of the sample protein. A user may input the protein structure data of the sample protein into the electronic device, or another device may send the protein structure data of the sample protein to the electronic device, so that the electronic device obtains the protein structure data of the sample protein.

A small drug molecule is a substance whose molecular weight is less than a threshold (for example, the molecular weight is less than 500) and may be configured for making drugs. In some embodiments, the small drug molecule may be referred to as a small molecule for short. The small drug molecule has a certain spatial structure (for example, a three-dimensional structure), and the small molecule structure data of the sample small drug molecule may be used to represent a spatial structure of the sample small drug molecule. A user may input the small molecule structure data of the sample small drug molecule into the electronic device, or another device may send the small molecule structure data of the sample small drug molecule to the electronic device, so that the electronic device may obtain the small molecule structure data of the sample small drug molecule.

In some embodiments, one sample protein and one sample small drug molecule may be referred to as a sample pair. In the sample pair, a function of the sample protein is affected by interaction between the sample small drug molecule and the sample protein, and an effect of treating diseases may be achieved through a series of related reactions. The interaction between the sample small drug molecule and the sample protein may be measured by using a binding affinity (Binding Affinity). For example, the binding affinity is configured for measuring a strength (also referred to as a binding strength) of interaction between a protein and a small drug molecule. The binding affinity includes data types such as an inhibition constant (referred to as a Ki value for short), a dissociation constant (referred to as a Kd value for short), and a Michaelis constant (referred to as a Km value for short). For example, the Ki value is configured for measuring an action form of the sample small drug molecule in inhibiting a protein function, and the Kd value is configured for measuring an action form of the sample small drug molecule in activating a protein function. In general, the sample pair is used as an example, and the binding affinity is configured for measuring the binding strength of the sample protein and the sample small drug molecule. A greater binding affinity indicates a higher binding strength between the sample protein and the sample small drug molecule and a greater druggability potential of the small drug molecule.

In some embodiments, for a sample pair, a binding affinity between a sample protein and a sample small drug molecule in the sample pair may be manually labeled to obtain a sample labeling result of the sample pair, so that the electronic device obtains the sample labeling result of the sample pair. The sample labeling result is obtained through labeling and is the binding affinity between the sample protein and the sample small drug molecule. In other words, the sample labeling result is obtained through labeling and is configured for representing the binding affinity between the sample protein and the sample small drug molecule. A quantity of the sample pairs is at least one, sample proteins and/or sample small drug molecules in any two sample pairs are different, and the sample proteins and/or sample small drug molecules refer to: at least one of sample proteins and sample small drug molecules.

Operation 202: Call a neural network model to determine structure data of a plurality of target complex conformations based on the protein structure data and the small molecule structure data.

The protein is used as a receptor, and the small drug molecule is used as a ligand. The ligand and the receptor form a complex conformation through binding, and the complex conformation may also be referred to as a binding pose (Binding Pose). A three-dimensional structure of the complex conformation is closely related to a three-dimensional structure of the ligand and a three-dimensional structure of the receptor. In some embodiments, a three-dimensional structure of the sample protein is represented by the protein structure data, and a three-dimensional structure of the sample small drug molecule is represented by the small molecule structure data. Based on the protein structure data and the small molecule structure data, three-dimensional structures of target complex conformations formed by binding of the sample protein and the sample small drug molecule may be obtained through prediction, and the three-dimensional structure of any target complex conformation is represented by the structure data of the target complex conformation. A target complex conformation is a structure obtained through prediction and formed by the binding of the sample protein and the sample small drug molecule. For example, the target complex conformation may be determined by the neural network model.

The neural network model in some embodiments may be an initial network model. The initial network model is an untrained model, that is, a model structure, a size, and the like of the neural network model are the same as a model structure, a size, and the like of the initial network model. The model structure and the size of the initial network model are not limited herein. For example, the initial network model includes, but not limited to, a conformational feature extraction part, a feature fusion part, and a prediction part that are sequentially connected in series. For example, the initial network model may also include a data enhancement part. For functions of the parts of the initial network model, reference may be made to related descriptions below and details are not described herein again. In some embodiments, the neural network model may also be a model obtained by training the initial network model at least once according to the manner from operation 201 to operation 204, or a model obtained by training the initial network model at least once according to another training manner.

In some embodiments, operation 202 includes operation 2021 to operation 2023.

Operation 2021: Generate structure data of a plurality of candidate complex conformations based on the protein structure data and the small molecule structure data.

For example, the protein structure data and the small molecule structure data may be inputted into simulation software, the simulation software is called to predict three-dimensional structures of candidate complex conformations formed by the binding of the sample protein and the sample small drug molecule, and the structure data of the candidate complex conformations are outputted, so that the three-dimensional structures of the candidate complex conformations are represented by using the structure data of the candidate complex conformations. In some embodiments, the simulation software is docking software. The docking software may simulate and predict a position and an orientation of a small drug molecule in a protein. For a small drug molecule and a protein, since there may be a plurality of positions and orientations of the small drug molecule in the protein, one position and one orientation of the small drug molecule in the protein correspond to one docking manner of the small drug molecule and the protein. The docking manner forms a three-dimensional structure of one candidate complex conformation. Therefore, the docking software may obtain the three-dimensional structures of the plurality of candidate complex conformations by predicting various docking manners of the small drug molecule and the protein, and only a few of these candidate complex conformations may be close to a real binding situation of the small drug molecule and the protein.

In some embodiments, the protein structure data includes at least one type; and operation 2021 includes: for any type of protein structure data, generating structure data of a candidate complex conformation corresponding to the type of protein structure data based on the type of protein structure data and the small molecule structure data; and determining the structure data of the plurality of candidate complex conformations based on structure data of candidate complex conformations corresponding to various types of protein structure data.

Generally, a three-dimensional structure of a protein is flexible and changes dynamically in a physiological environment. During binding of the protein and a small drug molecule, the three-dimensional structure of the protein also changes accordingly based on a three-dimensional structure of the small drug molecule. Based on the foregoing reason, in some embodiments, for any sample protein, at least one type of protein structure data of the sample protein may be obtained to express various three-dimensional structures of the sample protein by using the at least one type of protein structure data. Through the various three-dimensional structures of the sample protein, three-dimensional structures of candidate complex conformations formed by the binding of the sample protein and the sample small drug molecule that are obtained through prediction are enriched, so that the candidate complex conformations more likely cover the real binding situation of the sample protein and the sample small drug molecule, thereby improving a possibility of correct docking between the sample protein and the sample small drug molecule.

For any type of protein structure data, the protein structure data and small molecule structure data may be inputted into the simulation software, and the simulation software is called to predict at least one candidate complex conformation formed after the sample protein and the sample small drug molecule are combined based on a three-dimensional structure of a sample protein corresponding to the protein structure data and a three-dimensional structure of a sample small drug molecule corresponding to the protein structure data, and structure data of the at least one candidate complex conformation corresponding to the protein structure data is outputted.

Since any type of protein structure data corresponds to structure data of at least one candidate complex conformation, the sample protein corresponds to at least one type of protein structure data. Therefore, structure data of various candidate complex conformations corresponding to various types of protein structure data may be collectively referred to as structure data of various (that is, a plurality of) candidate complex conformations corresponding to the sample protein.

Operation 2022: Call the neural network model to determine quality indicators of the candidate complex conformations based on the structure data of the candidate complex conformations, where the quality indicator of each candidate complex conformation is configured for indicating quality of the candidate complex conformation.

In some embodiments, the structure data of the candidate complex conformations may be inputted into the neural network model. The neural network model is used to score, based on the structure data of any candidate complex conformation, the quality of the candidate complex conformation, to obtain the quality indicator of the candidate complex conformation. In some embodiments, the quality indicator of the candidate complex conformation is data that is greater than or equal to 0 and less than or equal to 1. A larger similarity between a candidate complex conformation and the real binding situation of the sample protein and the sample small drug molecule indicates higher quality of the candidate complex conformation and a larger quality indicator of the candidate complex conformation.

In some embodiments, operation 2022 includes operation A1 to operation A3.

Operation A1: Determine graph structures of the candidate complex conformations based on the structure data of the candidate complex conformations, where the graph structure of any candidate complex conformation is configured for representing a spatial structure of the candidate complex conformation.

In some embodiments, based on the structure data of any candidate complex conformation, the graph structure of the candidate complex conformation may be determined, and the three-dimensional structure of the candidate complex conformation is expressed concisely and vividly by using the graph structure of the candidate complex conformation. The graph structure of the candidate complex conformation includes a plurality of nodes and a plurality of edges. There is at least one edge on any node and the node is connected to another node through any edge. There may or may not be an edge between any two nodes.

In some embodiments, the structure data of any candidate complex conformation includes atomic data of a plurality of atoms, and any one of the atoms is an atom in the sample protein or an atom in the sample small drug molecule; and the graph structure of the candidate complex conformation includes a plurality of nodes and a plurality of edges. In this case, operation A1 includes the following operations: for the candidate complex conformation, determining distance data between every two atoms based on the atomic data of the plurality of atoms included in the candidate complex conformation; determining the nodes included in the graph structure of the candidate complex conformation based on the atomic data of the atoms included in the candidate complex conformation; and for any two nodes included in the graph structure of the candidate complex conformation, in a case that the distance data between two atoms corresponding to the two nodes is less than a distance threshold, adding an edge between the two nodes.

In a case that the sample protein includes a plurality of atoms, the protein structure data includes atomic data of the plurality of atoms. In a case that the sample small drug molecule includes a plurality of atoms, the small molecule structure data includes atomic data of the plurality of atoms. Therefore, when at least one candidate complex conformation is obtained through the binding of the sample protein and the sample small drug molecule, structure data of the candidate complex conformation includes the atomic data of the atoms in the sample protein and the atomic data of the atoms in the sample small drug molecule. The atomic data of any atom includes three-dimensional coordinates of the atom, an atom type, and the like. An atom category (that is, type) represents that the atom belongs to an atom in the sample protein or the atom belongs to an atom in the sample small drug molecule.

For any two atoms included in any candidate complex conformation, distance data between the two atoms may be determined according to a distance formula between two points based on three-dimensional coordinates of the two atoms. Next, the atomic data of the atoms included in the candidate complex conformation is used as nodes included in the graph structure of the candidate complex conformation. In other words, nodes included in the graph structure of any candidate complex conformation are determined based on atomic data of atoms included in the candidate complex conformation. The atomic data of each atom corresponds to a node in the graph structure of the candidate complex conformation. Based on an atom type included in atomic data of an atom, a representation form of a node corresponding to the atomic data of the atom may be determined. When the atom type represents that the atom belongs to an atom in the sample protein, a corresponding node may be represented in a first form (such as black), and when the atom type represents that an atom belongs to an atom in the sample small drug molecule, a corresponding node may be represented in a second form (such as white), where the first form is different from the second form.

For any two nodes included in the graph structure of the candidate complex conformation, in a case that the distance data between two atoms corresponding to the two nodes is less than a distance threshold, an edge is added between the two nodes. In this way, it may be determined whether there is an edge between every two nodes in the graph structure of the candidate complex conformation, so that the graph structure of the candidate complex conformation is obtained. The distance threshold is not limited thereto. For example, the distance threshold is 6 angstroms (a unit is Å). Angstrom is a unit of length measurement, a full name is Angstrom, and 1 angstrom is equal to 0.1 nanometer.

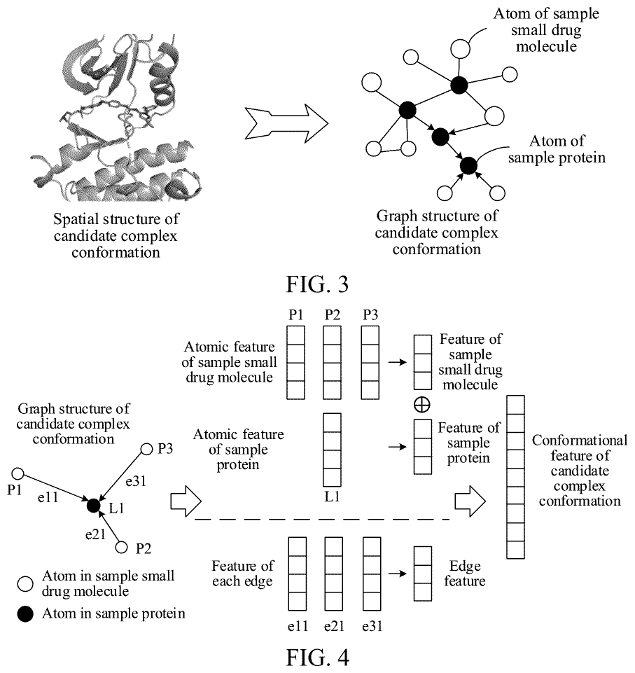

FIG. 3 is a schematic diagram of a spatial structure and a graph structure of a candidate complex conformation according to some embodiments. In some embodiments, a spatial structure of a candidate complex conformation may be converted into a graph structure of the candidate complex conformation. It may be seen from the graph structure of the candidate complex conformation that the graph structure of the candidate complex conformation includes a plurality of nodes and a plurality of edges, and any node is an atom of a sample small drug molecule or an atom of a sample protein. There is at least one edge on any node and the node is connected to another node through any edge. There may or may not be an edge between any two nodes.

Operation A2: Call the neural network model to determine conformational features of the candidate complex conformations based on the graph structures of the candidate complex conformations.

A graph structure of any candidate complex conformation is inputted into the neural network model, and through a conformational feature extraction part of the neural network model, a conformational feature of the candidate complex conformation is determined based on the graph structure of the candidate complex conformation.

In some embodiments, the graph structure of the candidate complex conformation includes the following information, where N represents a quantity of nodes, D represents content of a node, and L represents a quantity of edges.

GraphData (

x: content of each node (N*D)

edge_index: a list of edges on each node (L*2)

dist: a length of each edge (L*1)

coords: three-dimensional coordinates of each node (N*3)

node_type: a type of each node, where 0 represents that the node belongs to an atom in the sample small drug molecule, and 1 represents that the node belongs to an atom in the sample protein (N*1)

tar_lig_id: an identity document (ID) of a sample pair including the sample small drug molecule and the sample protein

pose_id: an identity document of the candidate complex conformation

Rank Index: an identity document of a candidate complex conformation set corresponding to a sample pair generated by simulation software, where the candidate complex conformation set includes candidate complex conformations corresponding to the sample pair

y: a sample labeling result

label: a labeled category of the candidate complex conformation).

For an edge between a node A and a node B, the edge belongs to both an edge on the node A and an edge on the node B. Therefore, a quantity of edges in the list of edges (that is, a quantity of edges on each node) is two times of the quantity of edges.

For any node, the conformational feature extraction part in the neural network model may perform a pooling operation on at least one of the content of the node, the three-dimensional coordinates of the node, the type of the node, and the like, to extract a feature of the node. In some embodiments, features of the nodes are used as the conformational feature of the candidate complex conformation.

In some embodiments, for any edge, the conformational feature extraction part may perform a pooling operation on at least one of respective content, three-dimensional coordinates, types, and the like of two nodes at both ends of the edge, and/or a length of the edge, to extract a feature of the edge. In other words, for any edge, the conformational feature extraction part may perform a pooling operation on at least one of first reference information and a length of the edge. The first reference information includes at least one of respective content, three-dimensional coordinates, types, and the like of two nodes at both ends of the edge. In some embodiments, features of edges are used as the conformational feature of the candidate complex conformation. In some embodiments, features of nodes and features of edges are used as the conformational feature of the candidate complex conformation.

In some embodiments, the conformational feature extraction part may perform feature extraction by using a multi-category pooling operation, to obtain the conformational feature of the candidate complex conformation. For example, when a type of a node represents that the node belongs to an atom in the sample small drug molecule or the node belongs to an atom in the sample protein, and feature extraction is performed by using a multi-category pooling operation, for any atom in the sample small drug molecule or any atom in the sample protein, the multi-category pooling operation is performed on content of the atom, three-dimensional coordinates of the atom, and the like, to obtain an atomic feature of the atom, and the atomic feature of the atom corresponds to the feature of the node. Afterwards, the conformational feature of the candidate complex conformation is obtained based on the features of the nodes.

For another example, the graph structure of the candidate complex conformation includes a type of an edge. The type of the edge may represent three types of edges, which are respectively an edge between two atoms in the sample small drug molecule, an edge between two atoms in the sample protein, and an edge between an atom in the sample small drug molecule and an atom in the sample protein. For any of the foregoing three types of edges, a pooling operation may be performed on at least one of respective content, three-dimensional coordinates, types, and the like of two nodes at both ends of the edge, and/or a length of the edge, to obtain the feature of the edge. In other words, a pooling operation is performed on at least one of second reference information and a length of the edge, to obtain the feature of the edge. The second reference information includes at least one of respective content, three-dimensional coordinates, types, and the like of two nodes at both ends of the edge. Afterwards, the conformational feature of the candidate complex conformation is obtained based on the features of the edges.

FIG. 4 is a schematic diagram of extracting a conformational feature of a candidate complex conformation according to some embodiments. The graph structure of the candidate complex conformation includes atoms P1 to P3 in the sample small drug molecule, an atom L1 in the sample protein, an edge e11 between P1 and L1, an edge e21 between P2 and L1, and an edge e31 between P3 and L1. The conformational feature extraction part may extract an atomic feature of the sample small drug molecule (that is, atomic features of P1 to P3), an atomic feature of the sample protein (that is, an atomic feature of L1), and features of the edges (that is, features of e11, e21 and e31). The atomic features of P1 to P3 are used as a feature of the sample small drug molecule, the atomic feature of L1 is used as a feature of the sample protein, and the features of e11, e21 and e31 are used as edge features. The feature of the sample small drug molecule, the feature of the sample protein, and the edge features are used as the conformational feature of the candidate complex conformation.

An algorithm used by the conformational feature extraction part to extract the conformational feature of the candidate complex conformation is not limited herein. For example, the algorithm may be a PotentialNet algorithm, a SchNet algorithm, and the like. After the conformational feature extraction part extracts at least one of the features of the nodes and the features of the edges, statistics collection may be further performed on the graph structure of the candidate complex conformation to obtain a statistical feature. The statistical feature is configured for representing a quantity of nodes, a quantity of nodes corresponding to each node type, a quantity of edges corresponding to each edge type, and the like in the graph structure of the candidate complex conformation. Afterwards, the conformational feature of the candidate complex conformation is determined based on at least one of the statistical feature, the features of the nodes, and the features of the edges.

It may be understood that, the foregoing operation A1 and operation A2 are to first determine the graph structures of the candidate complex conformations based on the structure data of the candidate complex conformations, and then determine the conformational features of the candidate complex conformations based on the graph structures of the candidate complex conformations. During application, a three-dimensional convolutional neural network (3D CNN) may be used to directly perform feature extraction on the structure data of the candidate complex conformations, to obtain the conformational features of the candidate complex conformations.

Operation A3: Call the neural network model to determine the quality indicators of the candidate complex conformations based on the conformational features of the candidate complex conformations.

After the conformational feature extraction part of the neural network model extracts the conformational feature of the candidate complex conformation, the quality indicator of the candidate complex conformation may be determined based on the conformational feature of the candidate complex conformation through a data enhancement part of the neural network model.

In some embodiments, the data enhancement part of the neural network model includes a multi-layer perceptron (MLP) and an output layer. The multi-layer perceptron is configured for performing down-sampling processing on the conformational feature of the candidate complex conformation at least once to obtain a down-sampling feature, where a purpose of the down-sampling processing is to reduce a dimension of the conformational feature of the candidate complex conformation. The output layer is configured for mapping the down-sampling feature into the quality indicator of the candidate complex conformation, where the mapping processing may be at least one of linear mapping processing and nonlinear mapping processing.

In some embodiments, the neural network model may also perform feature extraction on the protein structure data to obtain a protein feature, and perform feature extraction on the small molecule structure data to obtain a small molecule feature. The multi-layer perceptron may aggregate at least one of the protein feature and the small molecule feature with the conformational feature of the candidate complex conformation to obtain an aggregated feature, and perform down-sampling processing on the aggregated feature at least once to obtain a down-sampling feature. Afterwards, the down-sampling feature is mapped into the quality indicator of the candidate complex conformation through the output layer.

Operation 2023: Call the neural network model to select a plurality of target complex conformations from the plurality of candidate complex conformations based on the quality indicators of the candidate complex conformations, to obtain the structure data of the plurality of target complex conformations.

In operation 2022, structure data of a plurality of candidate complex conformations may be generated based on the protein structure data and the small molecule structure data. Most complex conformations in the plurality of candidate complex conformations are different from the real structure formed by the binding of the sample protein and the sample small drug molecule, and only a few complex conformations are relatively consistent with the real structure. In a case that no selection is performed on the plurality of candidate complex conformations and the plurality of candidate complex conformations are directly used to train the binding affinity detection model, the model has an excessively large calculation amount and poor accuracy. If only one target complex conformation is selected from the plurality of candidate complex conformations, the target complex conformation may cover the real structure formed by the binding of the sample protein and the sample small drug molecule, or may not cover the real structure. As a result, the problem of poor accuracy may also exist.

Therefore, in some embodiments, some candidate complex conformations may be selected from the plurality of candidate complex conformations as the plurality of target complex conformations. Since the candidate complex conformations have structure data, and the plurality of target complex conformations are selected from the candidate complex conformations, the plurality of target complex conformations also have structure data, so that the structure data of the plurality of target complex conformations can be obtained. For example, a probability that one target complex conformation covers the real structure is 50%. In a case that three target complex conformations are selected from the plurality of candidate complex conformations, a probability that the three target complex conformations cover the real structure is 75%. Therefore, a probability of covering the real structure may be increased through the plurality of target complex conformations, so that the plurality of target complex conformations can express the real structure more accurately, thereby improving the accuracy of the binding affinity detection model.

After the quality indicators of the candidate complex conformations are obtained, a plurality of target complex conformations whose quality indicator is greater than a reference indicator may be selected from the plurality of candidate complex conformations to avoid manual selection of the target complex conformations. The reference indicator may be set data, for example, the reference indicator is 0.7. In some embodiments, the reference indicator may be a set quantity^{th} quality indicator after the quality indicators of the candidate complex conformations are sorted. For example, the quality indicators of the candidate complex conformations are sorted in descending order, and a fourth quality indicator is used as the reference indicator. In this case, candidate complex conformations corresponding to the first three quality indicators are selected from the plurality of candidate complex conformations as the plurality of target complex conformations.

In some embodiments, labeled categories of the candidate complex conformations may be determined, where the labeled category of any candidate complex conformation is configured for representing whether the candidate complex conformation is similar to a benchmark complex conformation, and the benchmark complex conformation is a real structure formed by the binding of the sample protein and the sample small drug molecule. After the quality indicators of the candidate complex conformations are obtained, a plurality of candidate complex conformations that are similar to the benchmark complex conformation may be first selected from the plurality of candidate complex conformations, and a plurality of target complex conformations whose quality indicator is greater than the reference indicator are then selected from the plurality of candidate complex conformations that are similar to the benchmark complex conformation. A manner of determining the labeled categories of the candidate complex conformations is similar to a manner of determining labeled categories of the target complex conformations mentioned below, and details are not described herein.

Operation 203: Call the neural network model to determine a sample prediction result based on the structure data of the target complex conformations.

The sample prediction result is obtained through prediction and is the binding affinity between the sample protein and the sample small drug molecule. In other words, the sample prediction result is obtained through prediction and is configured for representing the binding affinity between the sample protein and the sample small drug molecule. The binding affinity between the sample protein and the sample small drug molecule may be predicted through the neural network model based on the structure data of the target complex conformations, to obtain the sample prediction result. A larger sample prediction result represents a stronger predicted binding affinity between the sample protein and the sample small drug molecule and a greater druggability potential of the sample small drug molecule.

In some embodiments, operation 203 includes operation 2031 to operation 2032.

Operation 2031: Call the neural network model to determine weights of the target complex conformations based on the conformational features of the target complex conformations, where the conformational feature of any target complex conformation is determined based on the structure data of the target complex conformation.

In some embodiments, the neural network model includes a feature fusion part. The feature fusion part may determine, based on the conformational feature of any target complex conformation, a weight of the target complex conformation. Therefore, the weight of the target complex conformation is related to the conformational feature of the target complex conformation. Operation A1 and operation A2 have mentioned the manner of determining the conformational feature of the candidate complex conformation based on the structure data of the candidate complex conformation. Since the target complex conformation is selected from the plurality of candidate complex conformations, a manner of determining the conformational feature of the target complex conformation is similar to the manner of determining the conformational feature of the candidate complex conformation, and details are not described herein again.

In some embodiments, operation 2031 includes: calling the neural network model to determine weighted conformational features of the target complex conformations based on a reference weight and the conformational features of the target complex conformations; and calling the neural network model to perform normalization processing on the weighted conformational features of the target complex conformations to obtain the weights of the target complex conformations.

The feature fusion part in the neural network model includes the reference weight, and the reference weight is a set vector or is obtained by continuously adjusting the set vector. That is, the reference weight is a learnable vector. The feature fusion part may perform weighted fusion on the reference weight and the conformational feature of any target complex conformation to obtain a weighted fusion result of the target complex conformation, and obtain a weighted conformational feature of the target complex conformation based on the weighted fusion result of the target complex conformation.

In some embodiments, the weighted conformational feature of the target complex conformation is determined according to Formula (1) shown below.

$${s}_{i} = f ( q {x}_{i} ) \qquad Formula ( 1 )$$

sᵢ represents a weighted conformational feature of an i^{th} target complex conformation. q represents the reference weight, xᵢ represents a conformational feature of the i^{th} target complex conformation, qxᵢ represents a weighted fusion result of the i^{th} target complex conformation, and f( ) represents a conversion function in a case that the weighted fusion result is converted into the weighted conformational feature of the i^{th} target complex conformation.

Next, the feature fusion part may perform normalization processing on the weighted conformational features of the target complex conformations according to a normalization function, to obtain the weights of the target complex conformations. The normalization function is not limited herein. For example, the normalization function is a Softmax function. The Softmax function may compress the weighted conformational feature of the target complex conformation to a range from 0 to 1, to obtain the weight of the target complex conformation. In some embodiments, the weight of the target complex conformation is determined according to Formula (2) shown below.

$${α}_{i} = softmax ( {s}_{i} ) = \frac{exp ( {s}_{i} )}{Σ exp ( {s}_{i} )} \qquad Formula ( 2 )$$

αᵢ represents a weight of the i^{th} target complex conformation, softmax represents a function symbol of the normalization function, sᵢ represents the weighted conformational feature of the i^{th} target complex conformation, exp represents an exponential function using a natural constant e as the base, and Σ represents a function symbol of a summation function.

The foregoing Formula (1) and Formula (2) determine a weight of a single target complex conformation to all target complex conformations through an attention mechanism. Therefore, the feature fusion part may include an attention network, and the attention network may be a self-attention network or a multi-head attention (Multi-Head Attention) network. In some embodiments, the reference weight is obtained through learning based on the attention mechanism.

Operation 2032: Call the neural network model to determine the sample prediction result based on the conformational features and the weights of the target complex conformations.

The feature fusion part in the neural network model may determine the sample prediction result based on the conformational features of the target complex conformations and the weights of the target complex conformations.

In some embodiments, operation 2032 includes: calling the neural network model to perform weighted calculation based on the conformational features and the weights of the target complex conformations to obtain a weighted fusion feature; and calling the neural network model to determine the sample prediction result based on the weighted fusion feature.

The feature fusion part in the neural network model may perform weighted summation on the conformational features and the weights of the target complex conformations, to obtain the weighted fusion feature. In some embodiments, the weighted fusion feature is determined according to Formula (3) shown below.

$$a = {\sum }_{i}^{N} {α}_{i} {x}_{i} \qquad Formula ( 3 )$$

a represents a weighted fusion feature, N represents a quantity of the target complex conformations, at represents the weight of the i^{th} target complex conformation, and xᵢ represents the conformational feature of the i^{th} target complex conformation.

Next, the sample prediction result is determined based on the weighted fusion feature through a prediction part of the neural network model. In some embodiments, the prediction part of the neural network model includes a multi-layer perceptron and an output layer. The multi-layer perceptron is configured for performing down-sampling processing on the weighted fusion feature at least once to obtain a down-sampling feature, where a purpose of the down-sampling processing is to reduce a dimension of the weighted fusion feature. The output layer is configured for mapping the down-sampling feature into the sample prediction result, where the mapping processing may be at least one of linear mapping processing and nonlinear mapping processing.

By determining the weights of the target complex conformations and performing weighted fusion on the conformational features of the target complex conformations by using the weights of the target complex conformations, the accuracy of the weighted fusion feature may be improved, thereby improving the accuracy of the sample prediction result.

In some embodiments, the neural network model may also perform feature extraction on the protein structure data to obtain a protein feature, and perform feature extraction on the small molecule structure data to obtain a small molecule feature. The multi-layer perceptron may aggregate at least one of the protein feature and the small molecule feature with the weighted fusion feature to obtain an aggregated feature, and perform down-sampling processing on the aggregated feature at least once to obtain a down-sampling feature. Afterwards, the down-sampling feature is mapped into the sample prediction result through the output layer.

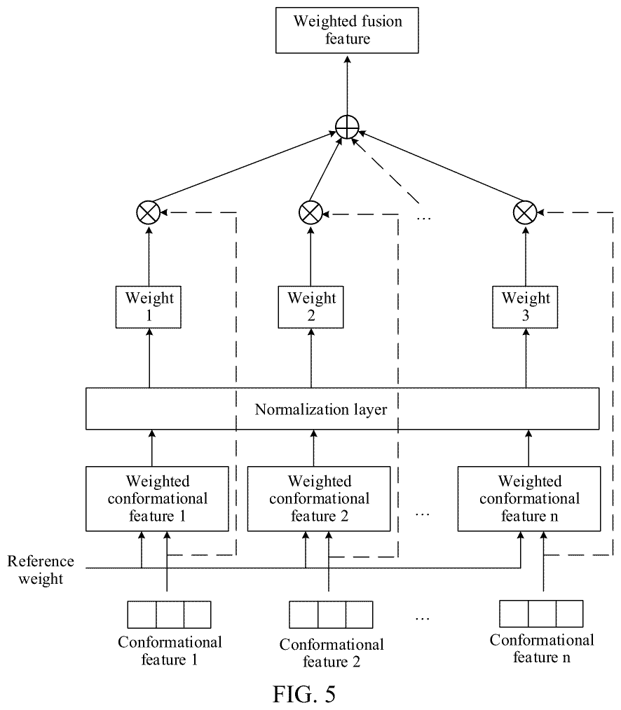

FIG. 5 is a schematic diagram of determining a weighted fusion feature according to some embodiments. In some embodiments, there are conformational features of n (n is a positive integer) target complex conformations, which are respectively denoted as a conformational feature 1 to a conformational feature n. Based on the reference weight and the conformational feature 1 to the conformational feature n, a weighted conformational feature 1 to a weighted conformational feature n may be obtained. Normalization processing is performed on the weighted conformational feature 1 to the weighted conformational feature n by using a normalization layer, to obtain a weight 1 to a weight n. By multiplying the weight 1 and the conformational feature 1, multiplying a weight 2 and a conformational feature 2, and so on, n multiplication results are obtained, and a weighted fusion feature is obtained by adding the n multiplication results.

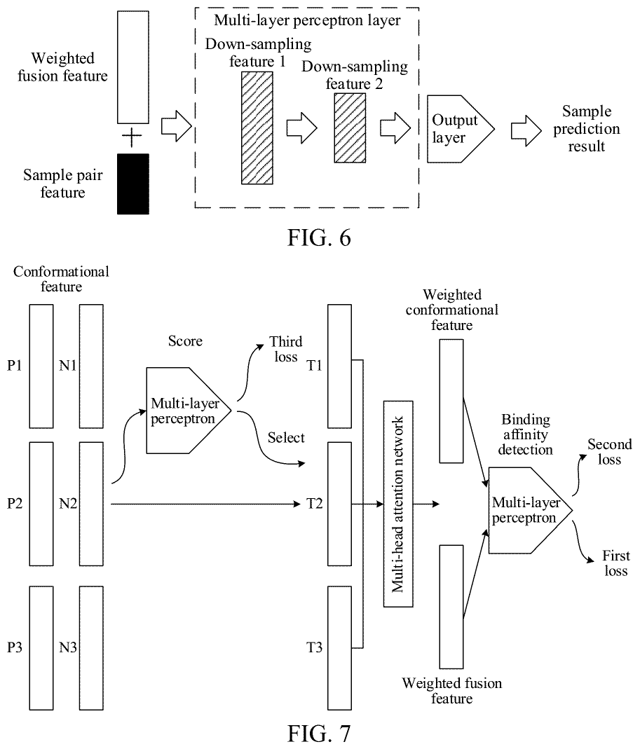

FIG. 6 is a schematic diagram of determining a sample prediction result according to some embodiments. In some embodiments, the weighted fusion feature may be spliced with a sample pair feature and then inputted into the multi-layer perceptron, where the sample pair feature include the protein feature and the small molecule feature. The multi-layer perceptron aggregates the protein feature, the small molecule feature, and the weighted fusion feature to obtain an aggregated feature, perform first down-sampling processing on the aggregated feature to obtain a down-sampling feature 1, and then perform second down-sampling processing on the down-sampling feature 1 to obtain a down-sampling feature 2. Afterwards, the down-sampling feature 2 is mapped into the sample prediction result through the output layer.

Operation 204: Train the neural network model by using the sample prediction result and the sample labeling result to obtain a binding affinity detection model.

The binding affinity detection model is configured for detecting a binding affinity between a target protein and a target small drug molecule. The binding affinity detection model may be applied in a preclinical research and development scenario of drug design, for example, applied in scenarios such as discovery of hit compounds and optimization of lead compounds. During training of the binding affinity detection model, the model may learn a binding mode between a protein and a small drug molecule, so that the binding affinity between the target protein and the target small drug molecule can be detected, further biological experimental verification may be performed on a target small drug molecule with a higher binding affinity, and a result of the biological experimental verification is used to provide feedback and guide to structural optimization of the target small drug molecule.

Through operation 201 to operation 203, the sample prediction result and the sample labeling result may be obtained. A loss of the neural network model may be calculated based on the sample prediction result and the sample labeling result, and the neural network model is trained once based on the loss of the neural network model, to obtain a trained neural network model.

In a case that the trained neural network model satisfies a training ending condition, the trained neural network model is used as the binding affinity detection model. The binding affinity detection model is configured for detecting a binding affinity between a target protein and a target small drug molecule in the manner of operation 801 to operation 803 mentioned below.

In a case that the trained neural network model does not satisfy the training ending condition, the trained neural network model is used as a neural network model for next training, and the neural network model is trained at least once again in the manner of operation 201 to operation 204 until the binding affinity detection model is obtained.

Satisfying the training ending condition is not limited herein. For example, the satisfying the training ending condition refers to that a quantity of training times of the neural network model reaches a set quantity of times (for example, 500 times). In some embodiments, the satisfying the training ending condition refers to that a difference between a loss of a neural network model obtained in last training and a loss of a neural network model obtained in next training is less than a threshold. In other words, in a case that a difference between a loss of a neural network model obtained in previous training of this training and a loss of a neural network model obtained in this training is less than the threshold, it may be considered that the training ending condition is satisfied.

In some embodiments, operation 204 includes operation 2041 to operation 2044.

Operation 2041: Determine a first loss based on the sample prediction result and the sample labeling result.

In some embodiments, the sample prediction result and the sample labeling result may be subtracted to obtain a difference between the sample prediction result and the sample labeling result, and the first loss is determined based on the difference. For example, at least one of square root operation, exponentiation operation, logarithm operation, and the like is performed on the difference to obtain the first loss.

Operation 2042: Determine prediction results of the target complex conformations based on the structure data of the target complex conformations, where the prediction result of each target complex conformation is obtained through prediction and is a binding affinity of the target complex conformation. In other words, the prediction result of the target complex conformation is obtained through prediction and is configured for representing the binding affinity of the target complex conformation. A greater prediction result of the target complex conformation represents a stronger binding affinity of the target complex conformation.

The prediction part of the neural network model may determine a prediction result of any target complex conformation based on the structure data of the target complex conformation. In some embodiments, the conformational feature of the target complex conformation may be first determined based on the structure data of the target complex conformation, and then, in a manner similar to “determining the sample prediction result based on the weighted fusion feature”, the prediction result of the target complex conformation is determined based on the conformational feature of the target complex conformation.

In some embodiments, the weights of the target complex conformations may be further determined based on the conformational features of the target complex conformations. The weighted conformational feature of the target complex conformation is determined based on the conformational feature and the weight of the target complex conformation, and then, in a manner similar to “determining the sample prediction result based on the weighted fusion feature”, the prediction result of the target complex conformation is determined based on the weighted conformational feature of the target complex conformation.

Operation 2043: For any target complex conformation, determine a second loss corresponding to the target complex conformation based on the sample labeling result and the prediction result of the target complex conformation.

In some embodiments, whether any target complex conformation is a positive sample or a negative sample may be distinguished based on a comparative learning strategy. In a case that the target complex conformation is a positive sample, a positive sample formula is used (refer to a formula for determining “the second loss corresponding to the target complex conformation” in case C1 below), and the second loss corresponding to the target complex conformation is determined based on the sample labeling result and the prediction result of the target complex conformation. In a case that the target complex conformation is a negative sample, a negative sample formula is used (refer to a formula for determining “the second loss corresponding to the target complex conformation” in case C2 below), and the second loss corresponding to the target complex conformation is determined based on the sample labeling result and the prediction result of the target complex conformation.

In some embodiments, operation 2043 includes operation B1 to operation B3.

Operation B1: Obtain structure data of a benchmark complex conformation.

The benchmark complex conformation is a real structure formed by the binding of the sample protein and the sample small drug molecule, and has a certain three-dimensional structure. In some embodiments, the structure data of the benchmark complex conformation may be obtained by analyzing the three-dimensional structure of the benchmark complex conformation, and the three-dimensional structure of the benchmark complex conformation is represented by using the structure data of the benchmark complex conformation.

Operation B2: Determine a labeled category of the target complex conformation based on the structure data of the benchmark complex conformation and the structure data of the target complex conformation, where the labeled category of the target complex conformation is configured for representing whether the target complex conformation is similar to the benchmark complex conformation.

In some embodiments, feature extraction may be performed on the structure data of the benchmark complex conformation to obtain a conformational feature of the benchmark complex conformation. Similarly, feature extraction may be performed on the structure data of any target complex conformation to obtain the conformational feature of the target complex conformation. For a manner of determining the conformational feature of the benchmark complex conformation and the conformational feature of the target complex conformation, reference may be made to the above-mentioned manner of determining the conformational feature of the candidate complex conformation. Implementation principles of the two manners are similar and details are not described herein again.

Next, based on the conformational feature of the benchmark complex conformation and the conformational feature of the target complex conformation, a similarity between the benchmark complex conformation and the target complex conformation may be determined according to a similarity formula. In a case that the similarity between the benchmark complex conformation and the target complex conformation is greater than a similarity threshold, it is determined that the labeled category of the target complex conformation is that the target complex conformation is similar to the benchmark complex conformation. In a case that the similarity between the benchmark complex conformation and the target complex conformation is not greater than the similarity threshold, it is determined that the labeled category of the target complex conformation is that the target complex conformation is not similar to the benchmark complex conformation. The similarity formula and the similarity threshold are not limited herein. For example, the similarity formula may be a distance formula, a cosine formula, a Jaccard formula, and the like. The similarity threshold is data greater than 0 and less than 1, and for example, the similarity threshold is 0.6.

In some embodiments, the structure data of the benchmark complex conformation includes atomic data of a plurality of atoms. Similarly, the structure data of any target complex conformation includes atomic data of a plurality of atoms. Since both the benchmark complex conformation and the target complex conformation are structures formed by the binding of the sample protein and the sample small drug molecule, the binding may change three-dimensional coordinates of an atom, but may not change a type of the atom, and a quantity of atoms. Therefore, atoms in the benchmark complex conformation are in a one-to-one correspondence with atoms in the target complex conformation.

Based on three-dimensional coordinates of any atom in the benchmark complex conformation and three-dimensional coordinates of a corresponding atom in the target complex conformation, a distance between the atom in the benchmark complex conformation and the corresponding atom in the target complex conformation may be calculated. Distances between the atoms in the benchmark complex conformation and the corresponding atoms in the target complex conformation are integrated to obtain a distance between the benchmark complex conformation and the target complex conformation. In some embodiments, the distance between the benchmark complex conformation and the target complex conformation may be obtained by performing root mean square deviation (RMSD) calculation based on three-dimensional coordinates of the atoms in the benchmark complex conformation and three-dimensional coordinates of the corresponding atoms in the target complex conformation.

In a case that the distance between the benchmark complex conformation and the target complex conformation is greater than a benchmark distance, it is determined that the labeled category of the target complex conformation is that the target complex conformation is not similar to the benchmark complex conformation. In a case that the distance between the benchmark complex conformation and the target complex conformation is not greater than the benchmark distance, it is determined that the labeled category of the target complex conformation is that the target complex conformation is similar to the benchmark complex conformation. The benchmark distance is not limited herein, and may be set based on application scenarios or experience. For example, the benchmark distance is 2 Å.

Operation B3: Determine the second loss corresponding to the target complex conformation based on the sample labeling result, and the prediction result and the labeled category of the target complex conformation.

In a case that the labeled category of the target complex conformation is that the target complex conformation is similar to the benchmark complex conformation, the target complex conformation is a positive sample, a positive sample formula may be used (refer to a formula for determining “the second loss corresponding to the target complex conformation” in case C1 below), and the second loss corresponding to the target complex conformation is determined based on the sample labeling result and the prediction result of the target complex conformation. In a case that the labeled category of the target complex conformation is that the target complex conformation is not similar to the benchmark complex conformation, the target complex conformation is a negative sample, a negative sample formula may be used (refer to a formula for determining “the second loss corresponding to the target complex conformation” in case C2 below), and the second loss corresponding to the target complex conformation is determined based on the sample labeling result and the prediction result of the target complex conformation.

In some embodiments, operation B3 includes two cases, respectively denoted as case C1 and case C2.

Case C1: In a case that the labeled category of the target complex conformation is configured for representing that the target complex conformation is similar to the benchmark complex conformation, determine the second loss corresponding to the target complex conformation based on a target difference between the prediction result of the target complex conformation and the sample labeling result.

When the target complex conformation is a positive sample, the target difference is determined according to Formula (4) shown below.

$$diff = pred - true , positive sample \qquad Formula ( 4 )$$

diff represents the target difference between the prediction result of the target complex conformation and the sample labeling result, pred represents the prediction result of the target complex conformation, and true represents the sample labeling result.

Next, at least one of square root operation, exponentiation operation, logarithm operation, and the like is performed on the target difference to obtain the second loss corresponding to the target complex conformation.

The second loss of the positive sample is determined in the manner of case C1, which makes a prediction result of the positive sample continuously approach the sample labeling result, and improves the accuracy of the prediction result of the positive sample, thereby improving the accuracy of the binding affinity detection model.

Case C2: In a case that the labeled category of the target complex conformation is configured for representing that the target complex conformation is not similar to the benchmark complex conformation, determine the second loss corresponding to the target complex conformation based on a maximum value of the target difference and a set value.

A magnitude of the set value is not limited herein. An example in which the set value is 0 is used. When the target complex conformation is a negative sample, the maximum value between the target difference and the set value may be determined according to Formula (5) shown below.

$$diff = max ( pred - true , 0 ) , negative sample \qquad Formula ( 5 )$$

diff represents the maximum value of the target difference and the set value, pred-true represents the target difference between the prediction result of the target complex conformation and the sample labeling result, pred represents the prediction result of the target complex conformation, and true represents the sample labeling result. max represents a function symbol of a maximum function.

At least one of square root operation, exponentiation operation, logarithm operation, and the like is performed on the maximum value of the target difference and the set value to obtain the second loss corresponding to the target complex conformation.

The second loss of a negative sample is determined in the manner of case C2, and when the prediction result of the negative sample is greater than the sample labeling result, a penalty is imposed on the negative sample, so that the prediction result of the negative sample is less than or equal to the sample labeling result. When the neural network model is subsequently trained based on the second loss corresponding to the negative sample, the trained neural network model may output a smaller prediction result for a negative sample, which improves a perception capability of the model for negative samples, and facilitates the model to distinguish positive samples and negative samples. Therefore, when the second loss of the negative sample is determined in the manner of case C2 and the binding affinity detection model is obtained by training based on the second loss corresponding to the negative sample, the binding affinity detection model has relatively high sensitivity to the target complex conformation and can more accurately distinguish positive samples and negative samples, so that the binding affinity detection model has relatively high accuracy.

Operation 2044: Train the neural network model to obtain the binding affinity detection model based on the first loss and second losses corresponding to the target complex conformations.

For example, the second losses corresponding to the target complex conformations may be added to obtain a total second loss. Weighted summation operation is performed on the first loss and the total second loss to obtain a loss of the neural network model, and the neural network model is trained based on the loss of the neural network model to obtain the binding affinity detection model.

In some embodiments, operation 204 includes operation 2045 to operation 2048.

Operation 2045: Determine a first loss based on the sample prediction result and the sample labeling result. For an implementation of operation 2045, reference may be made to the description of operation 2041 above, and details are not described herein again.

Operation 2046: Obtain labeled categories of the candidate complex conformations, where the labeled category of each candidate complex conformation is configured for representing whether the candidate complex conformation is similar to the benchmark complex conformation.

The labeled category of any candidate complex conformation may be determined based on the structure data of the benchmark complex conformation and the structure data of the candidate complex conformation. A manner of determining the labeled categories of the candidate complex conformations is similar to the manner of determining the labeled categories of the target complex conformations. Reference may be made to the description of operation B2 above, and details are not described herein again.

Operation 2047: For any candidate complex conformation, determine a third loss corresponding to the candidate complex conformation based on the labeled category and the quality indicator of the candidate complex conformation.

In some embodiments, in a case that the labeled category of any candidate complex conformation represents that the candidate complex conformation is similar to the benchmark complex conformation, it is determined that a labeled indicator of the candidate complex conformation is first data (such as 1), and based on the first data and the quality indicator of the candidate complex conformation, the third loss corresponding to the candidate complex conformation is calculated according to a cross-entropy loss function.

In a case that the labeled category of any candidate complex conformation represents that the candidate complex conformation is not similar to the benchmark complex conformation, it is determined that a labeled indicator of the candidate complex conformation is second data (such as 0), and based on the second data and the quality indicator of the candidate complex conformation, the third loss corresponding to the candidate complex conformation is calculated according to a cross-entropy loss function.

Operation 2048: Train the neural network model to obtain the binding affinity detection model based on the first loss and third losses corresponding to the candidate complex conformations.

For example, the third losses corresponding to the candidate complex conformations may be added to obtain a total third loss. Weighted summation operation is performed on the first loss and the total third loss to obtain a loss of the neural network model, and the neural network model is trained based on the loss of the neural network model to obtain the binding affinity detection model.

In some embodiments, according to Formula (6) shown below, weighted summation operation is performed on the first loss, the total second loss, and the total third loss to obtain a loss of the neural network model, and the neural network model is trained based on the loss of the neural network model to obtain the binding affinity detection model.

$${ℒ}_{l o s s} = {ℒ}_{M S E}^{( sample )} + α * {ℒ}_{M S E}^{( p o s e )} + β * {ℒ}_{B C E}^{( p o s e )} \qquad Formula ( 6 )$$

[symbol] ₗₒₛₛ represents the loss of the neural network model, [symbol] _{MSE}^{(sample)} represents the first loss, [symbol] _{MSE}^{(pose)} represents the total second loss, [symbol] _{BCE}^{(pose)} represents the total third loss, α represents a weight of the total second loss, and β represents a weight of the total third loss.

FIG. 7 is a schematic diagram of training a binding affinity detection model according to some embodiments. The binding affinity detection model may be obtained by training the neural network model. The neural network model includes two multi-layer perceptrons and a multi-head attention network.

In some embodiments, for a plurality of candidate complex conformations formed by the binding of the sample protein and the sample small drug molecule, labeled categories of the candidate complex conformations are determined. In a case that the labeled category of a candidate complex conformation is that the candidate complex conformation is similar to a benchmark complex conformation, the candidate complex conformation is a positive sample. In a case that the labeled category of a candidate complex conformation is that the candidate complex conformation is not similar to the benchmark complex conformation, the candidate complex conformation is a negative sample.

Feature extraction is performed on structure data of positive samples to obtain conformational features of the positive samples, which are respectively denoted as P1 to P3. Similarly, feature extraction is performed on structure data of negative samples to obtain conformational features of the positive samples, which are respectively denoted as N1 to N3. P1 to P3 and N1 to N3 are scored by using the multi-layer perceptron to obtain quality indicators of the candidate complex conformations. Based on the quality indicators of the candidate complex conformations, a plurality of target complex conformations are selected from the positive samples or from the positive samples and the negative samples.

Conformational features of the target complex conformations are respectively denoted as T1 to T3, and T1 to T3 are all inputted to the multi-head attention network. On one hand, the multi-head attention network outputs weighted conformational features of the target complex conformations. The weighted conformational features of the target complex conformations may be inputted into the multi-layer perceptron for binding affinity prediction, to obtain prediction results of the target complex conformations. The prediction result of each target complex conformation is obtained through prediction and is a binding affinity of the target complex conformation. On the other hand, the multi-head attention network outputs a weighted fusion feature. The weighted fusion feature may be inputted into the multi-layer perceptron for binding affinity prediction to obtain a sample prediction result. The sample prediction result is obtained through prediction and is a binding affinity between a sample protein and a sample small drug molecule.

In addition, a sample labeling result may also be obtained. The sample labeling result is obtained through labeling and is the binding affinity between the sample protein and the sample small drug molecule. On one hand, a first loss is determined based on the sample prediction result and the sample labeling result. On another hand, second losses corresponding to the target complex conformations are determined based on the sample labeling result and the prediction results of the target complex conformations. On still another hand, third losses corresponding to the candidate complex conformations are determined based on the labeled categories and the quality indicators of the candidate complex conformations.

Next, a loss of the neural network model is determined based on the first loss, the second losses corresponding to the target complex conformations, and the third losses corresponding to the candidate complex conformations. Based on the loss of the neural network model, the neural network model is trained to obtain the binding affinity detection model.

The information (including, but not limited to, user equipment information, user personal information, and the like), data (including, but not limited to, data for analysis, stored data, displayed data, and the like), and signals involved in this application all are authorized by the user or fully authorized by each party, and the collection, use, and processing of relevant data need to comply with relevant laws and regulations of relevant countries and regions. For example, the protein structure data of the sample protein and the small molecule structure data of the sample small drug molecule involved in this application are obtained with full authorization.

The foregoing method is to call the neural network model to determine the structure data of the plurality of target complex conformations based on the protein structure data of the sample protein and the small molecule structure data of the sample small drug molecule. Any target complex conformation is a structure obtained through prediction and formed by binding of a sample protein and a sample small drug molecule. Through a plurality of target complex conformations, a probability of covering a real structure formed by the binding of the sample protein and the sample small drug molecule may be improved. That is, the plurality of target complex conformations can more accurately express the real structure formed by the binding of the sample protein and the sample small drug molecule, so that a sample prediction result is more accurate when a neural network model is called to determine the sample prediction result based on structure data of the target complex conformations. In this way, when the neural network model is trained by using the sample prediction result to obtain a binding affinity detection model, the accuracy of the binding affinity detection model can be improved, thereby improving the accuracy of a binding affinity detection result.

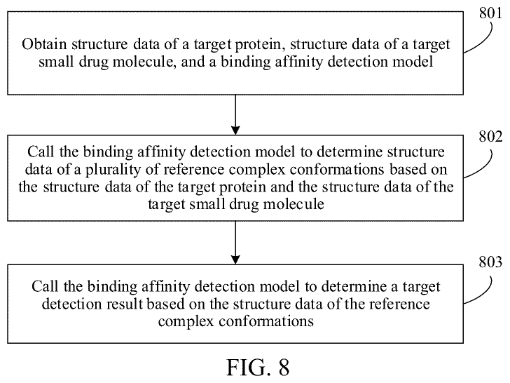

Some embodiments provide a binding affinity detection method, and the method may be applied in the foregoing implementation environment. A flowchart of a binding affinity detection method according to various embodiments shown in FIG. 8 is used as an example. For case of description, the terminal device 101 or the server 102 that performs the binding affinity detection method in some embodiments is referred to as an electronic device, and the method may be performed by the electronic device. As shown in FIG. 8, the method includes the following operations:

Operation 801: Obtain protein structure data of a target protein, structure data of a target small drug molecule, and a binding affinity detection model.

The binding affinity detection model is trained according to the method for training the binding affinity detection model related to FIG. 2. For a manner of obtaining the structure data of the target protein and the structure data of the target small drug molecule, reference may be made to the description of operation 201. Implementation principles of the two manners are similar and details are not described herein again.

Operation 802: Call the binding affinity detection model to determine structure data of a plurality of reference complex conformations based on the structure data of the target protein and the structure data of the target small drug molecule.

The reference complex conformation is a structure obtained through prediction and formed by binding of the target protein and the target small drug molecule. For description of operation 802, reference may be made to the description of operation 202 above. Implementation principles of the two operations are similar, and details are not described herein again.

In some embodiments, operation 802 includes operation 8021 to operation 8023.

Operation 8021: Generate structure data of a plurality of first complex conformations based on the structure data of the target protein and the structure data of the target small drug molecule. For description of operation 8021, reference may be made to the description of operation 2021 above. Implementation principles of the two operations are similar, and details are not described herein again.

Operation 8022: Call the binding affinity detection model to determine quality indicators of first complex conformations based on the structure data of the first complex conformations, where the quality indicator of each first complex conformation is configured for indicating quality of the first complex conformation. For description of operation 8022, reference may be made to the description of operation 2022 above. Implementation principles of the two operations are similar, and details are not described herein again.

Operation 8023: Call the binding affinity detection model to select a plurality of reference complex conformations from the plurality of first complex conformations based on the quality indicators of the first complex conformations, to obtain the structure data of the plurality of reference complex conformations. For description of operation 8023, reference may be made to the description of operation 2023 above. Implementation principles of the two operations are similar, and details are not described herein again.

Operation 803: Call the binding affinity detection model to determine a target detection result based on the structure data of the reference complex conformations.

The target detection result is a detected binding affinity between the target protein and the target small drug molecule. In other words, the target detection result is obtained through prediction and is configured for representing the binding affinity between the target protein and the target small drug molecule. For description of operation 803, reference may be made to the description of operation 203 above. Implementation principles of the two operations are similar, and details are not described herein again.

In some embodiments, operation 803 includes operation 8031 to operation 8032.

Operation 8031: Call the binding affinity detection model to determine weights of the reference complex conformations based on conformational features of the reference complex conformations, where the conformational feature of any reference complex conformation is determined based on the structure data of the reference complex conformation. For description of operation 8031, reference may be made to the description of operation 2031 above. Implementation principles of the two operations are similar, and details are not described herein again.

Operation 8032: Call the binding affinity detection model to determine the target detection result based on the conformational features and the weights of the reference complex conformations. For description of operation 8032, reference may be made to the description of operation 2032 above. Implementation principles of the two operations are similar, and details are not described herein again.

The information (including, but not limited to, user equipment information, user personal information, and the like), data (including, but not limited to, data for analysis, stored data, displayed data, and the like), and signals involved in this application all are authorized by the user or fully authorized by each party, and the collection, use, and processing of relevant data need to comply with relevant laws and regulations of relevant countries and regions. For example, the protein structure data of the target protein and the structure data of the target small drug molecule involved in this application are obtained with full authorization.

The binding affinity detection model in the foregoing method determines the structure data of the plurality of reference complex conformations based on the structure data of the target protein and the structure data of the target small drug molecule. Any reference complex conformation is a structure obtained through prediction and formed by binding of the target protein and the target small drug molecule. Through a plurality of reference complex conformations, a probability of covering a real structure formed by the binding of the target protein and the target small drug molecule may be improved. That is, the plurality of reference complex conformations can more accurately express the real structure formed by the binding of the target protein and the target small drug molecule, so that a target detection result is more accurate when the binding affinity detection model is called to determine the target detection result based on the structure data of the reference complex conformations. That is, the accuracy of a binding affinity detection result is improved.

The foregoing describes the method for training a binding affinity detection model and the binding affinity detection method provided in some embodiments from a perspective of method operations, and the following systematically describes a binding affinity detection process provided in some embodiments.

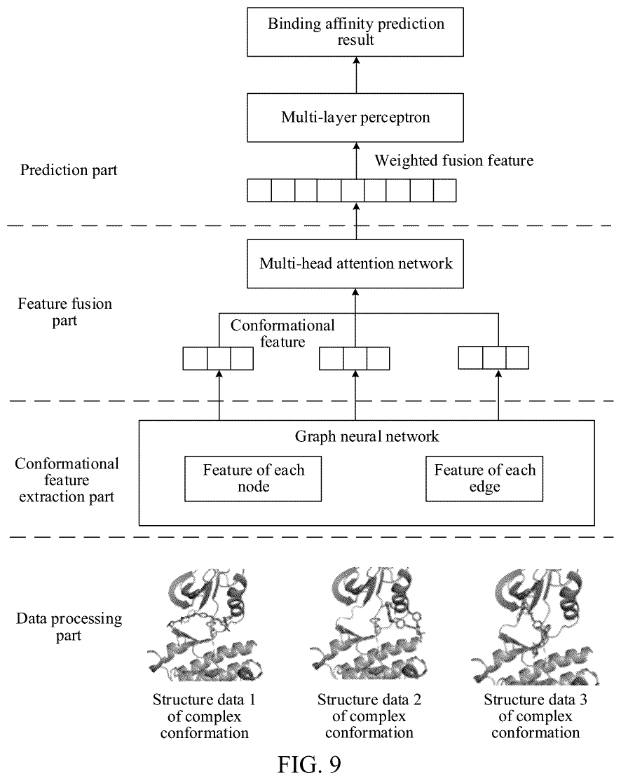

FIG. 9 is a schematic diagram of a binding affinity detection process according to some embodiments. The binding affinity detection process is divided into four parts, which are respectively a data processing part, a conformational feature extraction part, a feature fusion part, and a prediction part. The following separately describes the four parts:

Since a manner of determining a sample prediction result based on a sample protein and a sample small drug molecule is similar to a manner of determining a target detection result based on a target protein and a target small drug molecule, this embodiment briefly describes that a binding affinity prediction result is determined based on a protein and a small drug molecule.

The data processing part is configured for determining structure data of a plurality of complex conformations based on structure data of the protein and structure data of the small drug molecule, which are respectively denoted as structure data 1 of a complex conformation to structure data 3 of a complex conformation. Next, the structure data of the complex conformations are converted into graph structures of the complex conformations. In addition, the data processing part may also determine labeled categories of the complex conformations.

The conformational feature extraction part includes a graph neural network (GNN). The graph neural network may perform feature extraction on the graph structures of the complex conformations to obtain conformational features of the complex conformations. For any complex conformation, features of nodes and features of edges of the complex conformation may be extracted from the graph structure of the complex conformation. The conformational features of the complex conformations are obtained based on the features of the nodes and the features of the edges.

The feature fusion part includes a multi-head attention network, and the multi-head attention network is configured for determining a weighted fusion feature based on the conformational features of the complex conformations.

The prediction part includes a multi-layer perceptron, and the multi-layer perceptron is configured for determining the binding affinity prediction result based on the weighted fusion feature.

A public data set may be obtained in some embodiments. On one hand, an open source binding affinity detection model 1 (that is, a binding affinity detection model in the related art) is obtained. The binding affinity detection model may be a Gnina model. On the other hand, a binding affinity detection model is trained by using the public data set based on the method for training a binding affinity detection model related to FIG. 2, to respectively obtain a binding affinity detection model 2 and a binding affinity detection model 3. The binding affinity detection model 2 uses the first loss as the loss of the neural network model and is obtained through training by using the loss of the neural network model, while the binding affinity detection model 3 performs weighted summation on the first loss, the total second loss, and the total third loss to obtain the loss of the neural network model and is obtained through training by using the loss of the neural network model. Therefore, data enhancement is not performed in the binding affinity detection model 2, and data enhancement is performed in the binding affinity detection model 3.

According to some embodiments, the public data set is further used to test the detection accuracy of binding affinity detection models 1 to 3, to obtain results shown in Table 1 below.

TABLE 1

| Model | Pearson | RMSE | AUC |
|---|---|---|---|
| Binding affinity | 0.575 | / | 0.901 |
| detection model 1 | | | |
| Binding affinity | 0.574 | 1.661 | 0.857 |
| detection model 2 | | | |
| Binding affinity | 0.578 | 1.495 | 0.913 |
| detection model 3 | | | |

A pearson correlation coefficient (Pearson for short in Table 1) is a correlation degree between a true value of a binding affinity and a detected value of the binding affinity outputted by the binding affinity detection model. A higher Pearson indicates higher detection accuracy of the binding affinity detection models 1 to 3. A root mean square error (RMSE) is a root mean square error between the true value of the binding affinity and the detected value of the binding affinity outputted by the binding affinity detection model. A lower RMSE indicates higher detection accuracy of the binding affinity detection models 1 to 3. An area under curve (AUC) is an indicator for evaluating a labeled category of a complex conformation. A higher AUC indicates a more accurate labeled category of the complex conformation. It may be seen from Table 1 that, the detection accuracy of the binding affinity detection model that is trained based on the method for training a binding affinity detection model related to FIG. 2 is higher, and training the binding affinity detection model in a data enhancement manner can further improve the detection accuracy of the model.

A self-built data set may also be obtained in some embodiments. Since the self-built data set is prone to a data deviation compared with the public data set, an evaluation indicator of the self-built data set is easily excessively high. However, the self-built data set may cover more proteins (including mainstream proteins, new proteins, and the like), and binding of these proteins and small drug molecules better meets real application scenarios. In some embodiments, the self-built data set includes three proteins, which are respectively represented by Kinase, DUD-E, and Novel. A binding affinity detection model is trained by using the self-built data set based on the method for training a binding affinity detection model related to FIG. 2, to respectively obtain a binding affinity detection model 4 and a binding affinity detection model 5. Data enhancement is not performed in the binding affinity detection model 4, and data enhancement is performed in the binding affinity detection model 5.

According to some embodiments, the self-built data set is further used to test the detection accuracy of the binding affinity detection models 1, 4, and 5. For the three proteins Kinase, DUD-E, and Novel, Pearsons of the binding affinity detection models 1, 4, and 5 are respectively calculated to use the Pearson of each protein to locally evaluate the detection accuracy. In addition, for each binding affinity detection model, the Pearsons of the three proteins corresponding to the binding affinity detection model is used to calculate an average value to evaluate the detection accuracy as a whole. Obtained results are shown in Table 2 below.

TABLE 2

| | | | | Average |
|---|---|---|---|---|
| Model | Kinase | DUD-E | Novel | value |
| Binding affinity | 0.230 | 0.249 | 0.212 | 0.231 |
| detection model 1 | | | | |
| Binding affinity | 0.273 | 0.259 | 0.187 | 0.223 |
| detection model 4 | | | | |
| Binding affinity | 0.286 | 0.288 | 0.212 | 0.249 |
| detection model 5 | | | | |

It may be seen from Table 2 that, the detection accuracy of the binding affinity detection model that is trained based on the method for training a binding affinity detection model related to FIG. 2 is higher, and training the binding affinity detection model in a data enhancement manner can further improve the detection accuracy of the model.

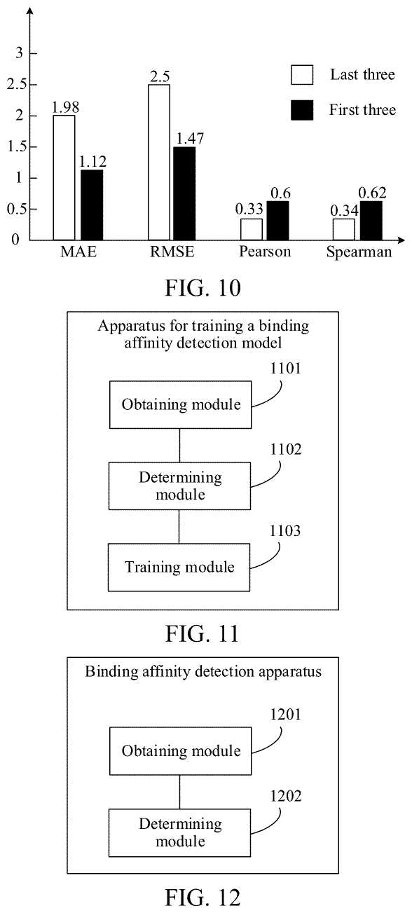

According to some embodiments, the binding affinity detection model 3 is also used to determine quality indicators of candidate complex conformations, and the quality indicators of the candidate complex conformations are sorted. On one hand, the first three candidate complex conformations after sorting are selected to test values of the binding affinity detection model 3 on four test indicators, namely, a mean absolute error (MAE), the RMSE, the Pearson, and a Spearman correlation coefficient (Spearman). On the other hand, the last three candidate complex conformations after sorting are selected to test values of the binding affinity detection model 3 on the four test indicators of the MAE, the RMSE, the Pearson, and the Spearman. Finally, a schematic diagram of comparison of test indicators shown in FIG. 10 is obtained.

It may be seen from FIG. 10 that, for the two test indicators of the MAE and the RMSE, test indicators of the last three candidate complex conformations after sorting (referred to as the last three) are both reduced by more than 0.8 (reduced by nearly 40%) compared with test indicators of the first three candidate complex conformations before sorting (referred to as the first three). For the two test indicators of the Pearson and the Spearman, compared with test indicators of the first three, test indicators of the last three are increased by about 0.3 (increased by nearly 80%). This indicates that data enhancement may improve the sensitivity of the model for complex conformations, so that the detection accuracy of the model is improved.

FIG. 11 is a schematic structural diagram of an apparatus for training a binding affinity detection model according to some embodiments. As shown in FIG. 11, the apparatus includes:
an obtaining module 1101, configured to obtain protein structure data of a sample protein, small molecule structure data of a sample small drug molecule, and a sample labeling result, where the sample labeling result is obtained through labeling and is a binding affinity between the sample protein and the sample small drug molecule (in other words, the sample labeling result is obtained through labeling and is configured for representing a binding affinity between the sample protein and the sample small drug molecule);
a determining module 1102, configured to call a neural network model to determine structure data of a plurality of target complex conformations based on the protein structure data and the small molecule structure data, where any target complex conformation is a structure obtained through prediction and formed by binding of the sample protein and the sample small drug molecule;
the determining module 1102, further configured to call the neural network model to determine a sample prediction result based on the structure data of the target complex conformations, where the sample prediction result is obtained through prediction and is the binding affinity between the sample protein and the sample small drug molecule (in other words, the sample prediction result is obtained through prediction and is configured for representing a binding affinity between the sample protein and the sample small drug molecule); and
a training module 1103, configured to train the neural network model by using the sample prediction result and the sample labeling result to obtain a binding affinity detection model, where the binding affinity detection model is configured for detecting a binding affinity between a target protein and a target small drug molecule.

In some embodiments, the determining module 1102 is configured to generate structure data of a plurality of candidate complex conformations based on the protein structure data and the small molecule structure data; call the neural network model to determine quality indicators of the candidate complex conformations based on the structure data of the candidate complex conformations, where the quality indicator of each candidate complex conformation is configured for indicating quality of the candidate complex conformation; and call the neural network model to select a plurality of target complex conformations from the plurality of candidate complex conformations based on the quality indicators of the candidate complex conformations, to obtain the structure data of the plurality of target complex conformations.

In some embodiments, the protein structure data includes at least one type; and
the determining module 1102 is configured to, for any type of protein structure data, generate structure data of a candidate complex conformation corresponding to the type of protein structure data based on the type of protein structure data and the small molecule structure data; and determine the structure data of the plurality of candidate complex conformations based on structure data of candidate complex conformations corresponding to various types of protein structure data.

In some embodiments, the determining module 1102 is configured to determine graph structures of the candidate complex conformations based on the structure data of the candidate complex conformations, where the graph structure of any candidate complex conformation is configured for representing a spatial structure of the candidate complex conformation; call the neural network model to determine conformational features of the candidate complex conformations based on the graph structures of the candidate complex conformations; and call the neural network model to determine the quality indicators of the candidate complex conformations based on the conformational features of the candidate complex conformations.

In some embodiments, the structure data of any candidate complex conformation includes atomic data of a plurality of atoms, and any one of the atoms is an atom in the sample protein or an atom in the sample small drug molecule; the graph structure of the candidate complex conformation includes a plurality of nodes and a plurality of edges; and
the determining module 1102 is configured to, for the candidate complex conformation, determine distance data between every two atoms based on the atomic data of the plurality of atoms included in the candidate complex conformation; use the atomic data of atoms included in the candidate complex conformation as the nodes included in the graph structure of the candidate complex conformation (in other words, determine the nodes included in the graph structure of the candidate complex conformation based on the atomic data of the atoms included in the candidate complex conformation); and for any two nodes included in the graph structure of the candidate complex conformation, in a case that the distance data between two atoms corresponding to the two nodes is less than a distance threshold, add an edge between the two nodes.

In some embodiments, the determining module 1102 is configured to call the neural network model to determine weights of the target complex conformations based on the conformational features of the target complex conformations, where the conformational feature of any target complex conformation is determined based on the structure data of the target complex conformation; and call the neural network model to determine the sample prediction result based on the conformational features and the weights of the target complex conformations.

In some embodiments, the determining module 1102 is configured to call the neural network model to determine weighted conformational features of the target complex conformations based on a reference weight and the conformational features of the target complex conformations; and call the neural network model to perform normalization processing on the weighted conformational features of the target complex conformations to obtain the weights of the target complex conformations.

In some embodiments, the determining module 1102 is configured to call the neural network model to perform weighted calculation based on the conformational features and the weights of the target complex conformations to obtain a weighted fusion feature; and call the neural network model to determine the sample prediction result based on the weighted fusion feature.

In some embodiments, the training module 1103 is configured to determine a first loss based on the sample prediction result and the sample labeling result; determine prediction results of the target complex conformations based on the structure data of the target complex conformations, where the prediction result of each target complex conformation is obtained through prediction and is a binding affinity of the target complex conformation (in other words, the prediction result of each target complex conformation is obtained through prediction and is configured for representing a binding affinity of the target complex conformation); for any target complex conformation, determine a second loss corresponding to the target complex conformation based on the sample labeling result and the prediction result of the target complex conformation; and train the neural network model to obtain the binding affinity detection model based on the first loss and second losses corresponding to the target complex conformations.

In some embodiments, the training module 1103 is configured to obtain structure data of a benchmark complex conformation, where the benchmark complex conformation is a real structure formed by the binding of the sample protein and the sample small drug molecule; determine a labeled category of the target complex conformation based on the structure data of the benchmark complex conformation and the structure data of the target complex conformation, where the labeled category of the target complex conformation is configured for representing whether the target complex conformation is similar to the benchmark complex conformation; and determine the second loss corresponding to the target complex conformation based on the sample labeling result, and the prediction result and the labeled category of the target complex conformation.

In some embodiments, the training module 1103 is configured to determine, in a case that the labeled category of the target complex conformation is configured for representing that the target complex conformation is similar to the benchmark complex conformation, the second loss corresponding to the target complex conformation based on a target difference between the prediction result of the target complex conformation and the sample labeling result; and determine, in a case that the labeled category of the target complex conformation is configured for representing that the target complex conformation is not similar to the benchmark complex conformation, the second loss corresponding to the target complex conformation based on a maximum value of the target difference and a set value.

In some embodiments, the training module 1103 is configured to determine a first loss based on the sample prediction result and the sample labeling result; obtain labeled categories of the candidate complex conformations, where the labeled category of each candidate complex conformation is configured for representing whether the candidate complex conformation is similar to the benchmark complex conformation; for any candidate complex conformation, determine a third loss corresponding to the candidate complex conformation based on the labeled category and the quality indicator of the candidate complex conformation; and train the neural network model to obtain the binding affinity detection model based on the first loss and third losses corresponding to the candidate complex conformations.

FIG. 12 is a schematic structural diagram of a binding affinity detection apparatus according to some embodiments. As shown in FIG. 12, the apparatus includes:
an obtaining module 1201, configured to obtain structure data of a target protein, structure data of a target small drug molecule, and a binding affinity detection model, where the binding affinity detection model is obtained through training based on the method for training a binding affinity detection model according to any one of the foregoing;
a determining module 1202, configured to call the binding affinity detection model to determine structure data of a plurality of reference complex conformations based on the structure data of the target protein and the structure data of the target small drug molecule, where the reference complex conformation is a structure obtained through prediction and formed by binding of the target protein and the target small drug molecule; and
the determining module 1202, further configured to call the binding affinity detection model to determine a target detection result based on the structure data of the reference complex conformations, where the target detection result is a detected binding affinity between the target protein and the target small drug molecule (in other words, the target detection result is obtained through detection and is configured for representing a binding affinity between the target protein and the target small drug molecule).

In some embodiments, the determining module 1202 is configured to generate structure data of a plurality of first complex conformations based on the structure data of the target protein and the structure data of the target small drug molecule; call the binding affinity detection model to determine quality indicators of the first complex conformations based on the structure data of the first complex conformations, where the quality indicator of each first complex conformation is configured for indicating quality of the first complex conformation; and call the binding affinity detection model to select a plurality of reference complex conformations from the plurality of first complex conformations based on the quality indicators of the first complex conformations, to obtain the structure data of the plurality of reference complex conformations.

In some embodiments, the determining module 1202 is configured to call the binding affinity detection model to determine weights of the reference complex conformations based on conformational features of the reference complex conformations, where the conformational feature of any reference complex conformation is determined based on the structure data of the reference complex conformation; and call the binding affinity detection model to determine the target detection result based on the conformational features and the weights of the reference complex conformations.

A person skilled in the art would understand that the above “modules” could be implemented by hardware logic, a processor or processors executing computer software code, or a combination of both. The “modules” may also be implemented in software stored in a memory of a computer or a non-transitory computer-readable medium, where the instructions of each module are executable by a processor to thereby cause the processor to perform the respective operations of the corresponding module. It may be understood that, when the apparatuses provided in FIG. 11 and FIG. 12 implement functions thereof, the foregoing embodiments are merely described by using division of various functional modules as an example. During actual application, the foregoing functions may be allocated to and completed by different functional modules as required. That is, the internal structure of the device is divided into different functional modules, to complete all or some of the functions described above. In addition, the apparatus and method embodiments provided in the foregoing embodiments belong to the same conception. For specific implementation processes and technical effects of the apparatus embodiments, reference may be made to the method embodiments (for example, for a specific implementation process and technical effects of the apparatus provided in FIG. 11, reference may be made to the method embodiment corresponding to FIG. 2, and for a specific implementation process and technical effects of the apparatus provided in FIG. 12, reference may be made to the method embodiment corresponding to FIG. 8), and details are not described herein again.

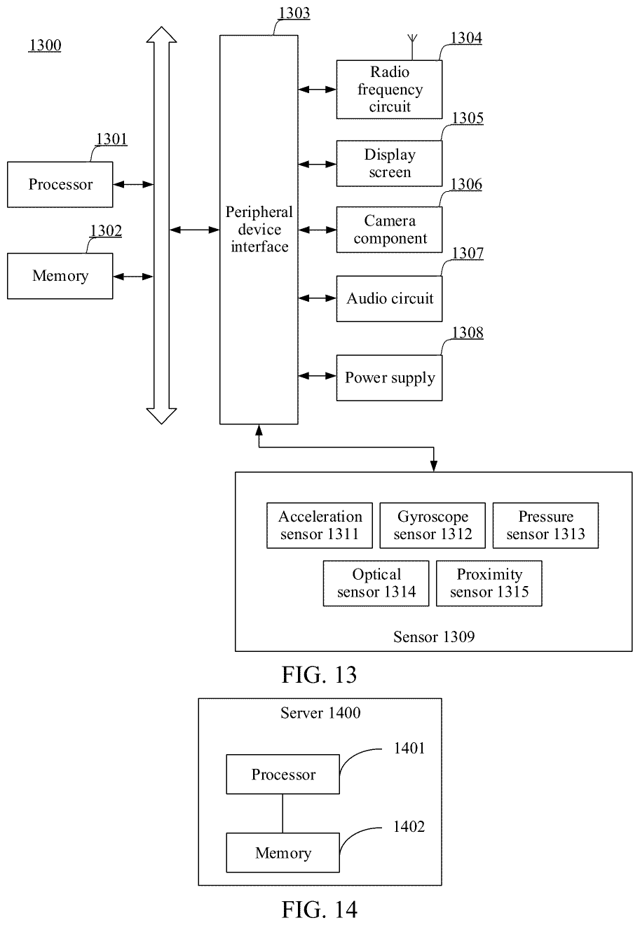

FIG. 13 is a structural block diagram of a terminal device 1300 according to some embodiments. The terminal device 1300 includes: a processor 1301 and a memory 1302.

The processor 1301 may include one or more processing cores, such as a 4-core processor or an 8-core processor. The processor 1301 may be implemented by using at least one hardware form of a digital signal processor (DSP), a field-programmable gate array (FPGA), and a programmable logic array (PLA). The processor 1301 may also include a main processor and a coprocessor. The main processor is a processor configured to process data in an active state, also referred to as a central processing unit (CPU). The coprocessor is a low-power consumption processor configured to process data in a standby state. In some embodiments, the processor 1301 may be integrated with a graphics processing unit (GPU). The GPU is configured to render and draw content that needs to be displayed on a display screen. In some embodiments, the processor 1301 may also include an artificial intelligence (AI) processor. The AI processor is configured to process a computing operation related to machine learning.

The memory 1302 may include one or more computer-readable storage media. The computer-readable storage medium may be non-transitory (also referred to as non-temporary). The memory 1302 may also include a high-speed random access memory and a non-volatile memory, for example, one or more disk storage devices and flash storage devices. In some embodiments, the non-transitory computer-readable storage medium in the memory 1302 is configured to store at least one computer program, and the at least one computer program is configured to be executed by the processor 1301 to implement the method for training a binding affinity detection model or the binding affinity detection method provided in various method embodiments.

In some embodiments, the terminal device 1300 may further include: a peripheral device interface 1303 and at least one peripheral device. The processor 1301, the memory 1302, and the peripheral device interface 1303 may be connected through a bus or a signal cable. Each peripheral device may be connected to the peripheral device interface 1303 through a bus, a signal cable, or a circuit board. Specifically, the peripheral device includes: at least one of a radio frequency circuit 1304, a display screen 1305, a camera component 1306, an audio circuit 1307, and a power supply 1308.

The peripheral device interface 1303 may be configured to connect at least one input/output (I/O)-related peripheral device to the processor 1301 and the memory 1302. In some embodiments, the processor 1301, the memory 1302, and the peripheral device interface 1303 are integrated on the same chip or the same circuit board. In some embodiments, any one or two of the processor 1301, the memory 1302, and the peripheral device interface 1303 may be implemented on a separate chip or circuit board, which is not limited in this embodiment.

The radio frequency circuit 1304 is configured to receive and transmit a radio frequency (RF) signal that is also referred to as an electromagnetic signal. The RF circuit 1304 communicates with a communication network and another communication device by using the electromagnetic signal. The RF circuit 1304 converts an electric signal into an electromagnetic signal for transmission, or converts a received electromagnetic signal into an electric signal. In some embodiments, the RF circuit 1304 includes: an antenna system, an RF transceiver, one or more amplifiers, a tuner, an oscillator, a digital signal processor, a codec chipset, a user identity module card, and the like. The RF circuit 1304 may communicate with other terminals through at least one wireless communication protocol. The wireless communication protocol includes but is not limited to: a world wide web, a metropolitan area network, an intranet, generations of mobile communication networks (2G, 3G, 4G, and 5G), a wireless local area network, and/or a wireless fidelity (Wi-Fi) network. In some embodiments, the RF circuit 1304 may also include a circuit related to near field communication (NFC), which is not limited herein.

The display screen 1305 is configured to display a user interface (UI). The UI may include a graph, a text, an icon, a video, and any combination thereof. When the display screen 1305 is a touch display screen, the display screen 1305 is further capable of collecting a touch signal on or above a surface of the display screen 1305. The touch signal may be inputted, as a control signal, to the processor 1301 for processing. In this case, the display screen 1305 may also be configured to provide a virtual button and/or a virtual keyboard, also referred to as a soft button and/or a soft keyboard. In some embodiments, there may be one display screen 1305 arranged on a front panel of the terminal device 1300. In some embodiments, there may be at least two display screens 1305 respectively arranged on different surfaces of the terminal device 1300 or in a folded design. In some embodiments, the display screen 1305 may be a flexible display screen arranged on a curved or folded surface of the terminal device 1300. Even further, the display screen 1305 may be arranged in a non-rectangular irregular pattern, that is, a special-shaped screen. The display screen 1305 may be prepared by using materials such as a liquid crystal display (LCD), an organic light-emitting diode (OLED), or the like.

The camera component 1306 is configured to collect images or videos. In some embodiments, the camera component 1306 includes a front-facing camera and a rear-facing camera. Generally, the front-facing camera is arranged on a front panel of the terminal, and the rear-facing camera is arranged on a rear surface of the terminal. In some embodiments, there are at least two rear-facing cameras, each being any one of a main camera, a depth-of-field camera, a wide-angle camera, and a telephoto camera, to achieve a background blurring function through fusion of the main camera and the depth-of-field camera, panoramic photo shooting and virtual reality (VR) shooting functions through fusion of the main camera and the wide-angle camera, or another fusion shooting function. In some embodiments, the camera component 1306 may further include a flash. The flash may be a single color temperature flash or a double color temperature flash. The double color temperature flash refers to a combination of a warm light flash and a cold light flash, and may be used for light compensation under different color temperatures.

The audio circuit 1307 may include a microphone and a speaker. The microphone is configured to collect sound waves from a user and an environment and convert the sound waves into electrical signals that are inputted to the processor 1301 for processing or inputted to the RF circuit 1304 for voice communication. For purposes of stereo collection or noise reduction, there may be a plurality of microphones, which are respectively arranged at different parts of the terminal device 1300. The microphone may be, in some embodiments, a microphone array or an omnidirectional acquisition microphone. The speaker is configured to convert the electrical signals from the processor 1301 or the RF circuit 1304 into sound waves. The speaker may be a conventional thin-film speaker or a piezoelectric ceramic speaker. When the speaker is a piezoelectric ceramic speaker, the speaker can not only convert an electric signal into sound waves audible to a human being, but also convert an electric signal into sound waves inaudible to the human being for ranging and other purposes. In some embodiments, the audio circuit 1307 may further include an earphone jack.

The power supply 1308 is configured to supply power to the components in the terminal device 1300. The power supply 1308 may be an alternating-current power supply, a direct-current power supply, a disposable battery, or a rechargeable battery. When the power supply 1308 includes a rechargeable battery, the rechargeable battery may be a wired charging battery or a wireless charging battery. The wired charging battery is a battery charged through a wired line, and the wireless charging battery is a battery charged through a wireless coil. The rechargeable battery may also be configured to support a fast charge technology.

In some embodiments, the terminal device 1300 further includes one or more sensors 1309. The one or more sensors 1309 include but are not limited to: an acceleration sensor 1311, a gyroscope sensor 1312, a pressure sensor 1313, an optical sensor 1314, and a proximity sensor 1315.

The acceleration sensor 1311 may detect accelerations on three coordinate axes of a coordinate system established by the terminal device 1300. For example, the acceleration sensor 1311 may be configured to detect components of a gravitational acceleration on the three coordinate axes. The processor 1301 may control the display screen 1305 to display the user interface in a lateral view or a longitudinal view based on a gravitational acceleration signal collected by the acceleration sensor 1311. The acceleration sensor 1311 may also be configured to collect game or user motion data.

The gyroscope sensor 1312 may detect a body direction and a rotation angle of the terminal device 1300, and the gyroscope sensor 1312 may collect a 3D action performed by a user on the terminal device 1300 in cooperation with the acceleration sensor 1311. The processor 1301 may implement the following functions based on the data collected by the gyroscope sensor 1312: motion sensing (for example, change the UI based on a tilt operation of the user), image stabilization during photographing, game control, and inertial navigation.

The pressure sensor 1313 may be arranged on a side frame of the terminal device 1300 and/or a lower layer of the display screen 1305. When the pressure sensor 1313 is arranged on the side frame of the terminal device 1300, a holding signal of the user to the terminal device 1300 may be detected, and the processor 1301 performs left and right hand recognition or a quick operation based on the holding signal collected by the pressure sensor 1313. When the pressure sensor 1313 is arranged on the lower layer of the display screen 1305, the processor 1301 controls an operable control on the UI based on a pressure operation of the user on the display screen 1305. The operable control includes at least one of a button control, a scroll-bar control, an icon control, and a menu control.

The optical sensor 1314 is configured to collect ambient light intensity. In an embodiment, the processor 1301 may control display brightness of the display screen 1305 based on the ambient light intensity collected by the optical sensor 1314. Specifically, when the ambient light intensity is high, the display brightness of the display screen 1305 is increased; and when the ambient light intensity is low, the display brightness of the display screen 1305 is decreased. In some embodiments, the processor 1301 may also dynamically adjust camera parameters of the camera component 1306 according to the ambient light intensity collected by the optical sensor 1314.

The proximity sensor 1315, also referred to as a distance sensor, is generally arranged on the front panel of the terminal device 1300. The proximity sensor 1315 is configured to collect a distance between the user and a front surface of the terminal device 1300. In an embodiment, when the proximity sensor 1315 detects that the distance between the user and the front surface of the terminal device 1300 is gradually reduced, the processor 1301 controls the display screen 1305 to switch from a screen-on state to a screen-off state. When the proximity sensor 1315 detects that the distance between the user and the front surface of the terminal device 1300 is gradually increased, the processor 1301 controls the display screen 1305 to switch from the screen-off state to the screen-on state.

A person skilled in the art may understand that the structure shown in FIG. 13 constitutes no limitation to the terminal device 1300, and the terminal device may include more or fewer components than those shown in the figure, or some components may be combined, or a different component arrangement may be used.

FIG. 14 is a schematic structural diagram of a server according to some embodiments. The server 1400 may vary greatly due to different configurations or performance, and may include one or more processors 1401 and one or more memories 1402. The one or more memories 1402 store at least one computer program, and the at least one computer program is loaded and executed by the one or more processors 1401 to implement the method for training a binding affinity detection model or the binding affinity detection method provided in the foregoing method embodiments. For example, the processor 1401 is a CPU. Certainly, the server 1400 may also have components such as a wired or wireless network interface, a keyboard, and an input/output interface to facilitate input/output. The server 1400 may also include other components for implementing device functions. Details are not described herein.

In some embodiments, a non-transitory computer-readable storage medium is further provided. The non-transitory computer-readable storage medium stores at least one computer program, and the at least one computer program is loaded and executed by a processor to cause an electronic device to implement any one of the foregoing method for training a binding affinity detection model or the foregoing binding affinity detection method.

In some embodiments, the non-transitory computer-readable storage medium may be a read-only memory (ROM), a random access memory (RAM), a compact disc read-only memory (CD-ROM), a magnetic tape, a floppy disk, an optical data storage device, and the like.

In some embodiments, a computer program or a computer program product is further provided. The computer program or the computer program product stores at least one computer program, and the at least one computer program is loaded and executed by a processor to cause an electronic device to implement any one of the foregoing method for training a binding affinity detection model or the foregoing binding affinity detection method.

It is to be understood that “a plurality of” mentioned in this specification refers to two or more. “And/or” describes an association relationship between associated objects and represents that three relationships may exist. For example, A and/or B may represent the following three cases: Only A exists, both A and B exist, and only B exists. The character “/” generally represents an “or” relationship between the associated objects.

The sequence numbers of the foregoing embodiments are merely for description purpose, and do not indicate the preference of the embodiments.

The foregoing embodiments are used for describing, instead of limiting the technical solutions of the disclosure. A person of ordinary skill in the art shall understand that although the disclosure has been described in detail with reference to the foregoing embodiments, modifications can be made to the technical solutions described in the foregoing embodiments, or equivalent replacements can be made to some technical features in the technical solutions, provided that such modifications or replacements do not cause the essence of corresponding technical solutions to depart from the spirit and scope of the technical solutions of the embodiments of the disclosure and the appended claims.

What is claimed is:

1. A method for training a binding affinity detection model, performed by an electronic device, comprising:
obtaining protein structure data of a sample protein, small molecule structure data of a sample small drug molecule, and a sample labeling result, the sample labeling result being obtained through labeling and indicating a binding affinity between the sample protein and the sample small drug molecule;
calling a neural network model to determine structure data of a plurality of target complex conformations based on the protein structure data and the small molecule structure data;
calling the neural network model to determine a sample prediction result based on the structure data of the target complex conformations; and
training the neural network model using the sample prediction result and the sample labeling result to obtain a binding affinity detection model to detect a binding affinity between a target protein and a target small drug molecule.

2. The method according to claim 1, wherein calling the neural network model to determine the structure data of the plurality of target complex conformations comprises:
generating structure data of a plurality of candidate complex conformations based on the protein structure data and the small molecule structure data;
calling the neural network model to determine quality indicators of the plurality of candidate complex conformations based on the structure data of the plurality of candidate complex conformations; and
calling the neural network model to select the plurality of target complex conformations from the plurality of candidate complex conformations based on the quality indicators to obtain the structure data of the plurality of target complex conformations.

3. The method according to claim 2, wherein the protein structure data comprises at least one type; and
wherein generating the structure data of a plurality of candidate complex conformations comprises:
for the at least one type of protein structure data, generating structure data of a candidate complex conformation corresponding to the type of protein structure data based on the type of protein structure data and the small molecule structure data; and
determining the structure data of the plurality of candidate complex conformations based on structure data of candidate complex conformations corresponding to various types of protein structure data.

4. The method according to claim 2, wherein calling the neural network model to determine quality indicators comprises:
determining graph structures of the plurality of candidate complex conformations based on the structure data of the plurality of candidate complex conformations, wherein the graph structure of any candidate complex conformation represents a spatial structure of the candidate complex conformation;
calling the neural network model to determine conformational features of the plurality of candidate complex conformations based on the graph structures of the plurality of candidate complex conformations; and
calling the neural network model to determine the quality indicators of the plurality of candidate complex conformations based on the conformational features of the plurality of candidate complex conformations.

5. The method according to claim 4, wherein the structure data of any of the plurality of candidate complex conformations comprises atomic data of a plurality of atoms, and any one of the atoms is an atom in the sample protein or an atom in the sample small drug molecule;
wherein the graph structure of the candidate complex conformation comprises a plurality of nodes and a plurality of edges; and
wherein determining the graph structures of the plurality of candidate complex conformations based on the structure data of the plurality of candidate complex conformations comprises:
for each candidate complex conformation, determining distance data between every two atoms based on the atomic data of the plurality of atoms comprised in each candidate complex conformation;
determining the nodes comprised in the graph structure of each candidate complex conformation based on the atomic data of the atoms comprised in each candidate complex conformation; and
for any two nodes comprised in the graph structure of each candidate complex conformation, based on the distance data between two atoms corresponding to the two nodes being less than a distance threshold, adding an edge between the two nodes.

6. The method according to claim 1, wherein calling the neural network model to determine the sample prediction result comprises:
calling the neural network model to determine weights of the plurality of target complex conformations based on the conformational features of the plurality of target complex conformations, the conformational feature of each target complex conformation being determined based on the structure data of each target complex conformation; and
calling the neural network model to determine the sample prediction result based on the conformational features and the weights of the plurality of target complex conformations.

7. The method according to claim 6, wherein calling the neural network model to determine the weights of the target complex conformations comprises:
calling the neural network model to determine weighted conformational features of the plurality of target complex conformations based on a reference weight and the conformational features of the plurality of target complex conformations; and
calling the neural network model to perform normalization processing on the weighted conformational features of the plurality of target complex conformations to obtain the weights of the plurality of target complex conformations.

8. The method according to claim 6, wherein calling the neural network model to determine the sample prediction result based on the conformational features and the weights of the plurality of target complex conformations comprises:
calling the neural network model to perform weighted calculation based on the conformational features and the weights of the plurality of target complex conformations to obtain a weighted fusion feature; and
calling the neural network model to determine the sample prediction result based on the weighted fusion feature.

9. The method according to claim 1, wherein training the neural network model using the sample prediction result and the sample labeling result comprises:
determining a first loss based on the sample prediction result and the sample labeling result;
determining prediction results of the plurality of target complex conformations based on the structure data of the plurality of target complex conformations, the prediction result of each target complex conformation being obtained through prediction and indicating a binding affinity of the target complex conformation;
for each target complex conformation, determining a second loss corresponding to the target complex conformation based on the sample labeling result and the prediction result of the target complex conformation; and
training the neural network model to obtain the binding affinity detection model based on the first loss and second losses corresponding to the target complex conformations.

10. The method according to claim 9, wherein determining the second loss corresponding to the target complex conformation based on the sample labeling result and the prediction result of the target complex conformation comprises:
obtaining structure data of a benchmark complex conformation, the benchmark complex conformation being a real structure formed by the binding of the sample protein and the sample small drug molecule;
determining a labeled category of the target complex conformation based on the structure data of the benchmark complex conformation and the structure data of the target complex conformation, the labeled category of the target complex conformation indicating whether the target complex conformation is similar to the benchmark complex conformation; and
determining the second loss corresponding to the target complex conformation based on the sample labeling result, and the prediction result and the labeled category of the target complex conformation.

11. The method according to claim 10, wherein determining the second loss corresponding to the target complex conformation based on the sample labeling result, and the prediction result and the labeled category of the target complex conformation comprises:
based on the labeled category of the target complex conformation indicating that the target complex conformation is similar to the benchmark complex conformation, determining the second loss corresponding to the target complex conformation based on a target difference between the prediction result of the target complex conformation and the sample labeling result; and
based on the labeled category of the target complex conformation indicating that the target complex conformation is not similar to the benchmark complex conformation, determining the second loss corresponding to the target complex conformation based on a maximum value of the target difference and a set value.

12. The method according to claim 1, wherein training the neural network model using the sample prediction result and the sample labeling result comprises:
determining a first loss based on the sample prediction result and the sample labeling result;
obtaining labeled categories of the plurality of candidate complex conformations, the labeled category of each candidate complex conformation indicating whether each candidate complex conformation is similar to the benchmark complex conformation;
for each candidate complex conformation, determining a third loss corresponding to the candidate complex conformation based on the labeled category and the quality indicator of the candidate complex conformation; and
training the neural network model to obtain the binding affinity detection model based on the first loss and the third loss corresponding to the plurality of candidate complex conformations.

13. An apparatus for training a binding affinity detection model, comprising:
at least one memory configured to store program code; and
at least one processor configured to read the program code and operate as instructed by the program code, the program code comprising:
obtaining code configured to cause at least one of the at least one processor to obtain protein structure data of a sample protein, small molecule structure data of a sample small drug molecule, and a sample labeling result;
determining code configured to cause at least one of the at least one processor to:
call a neural network model to determine structure data of a plurality of target complex conformations based on the protein structure data and the small molecule structure data;
call the neural network model to determine a sample prediction result based on the structure data of the target complex conformations, the sample prediction result being obtained through prediction and indicating the binding affinity between the sample protein and the sample small drug molecule; and
training code configured to cause at least one of the at least one processor to train the neural network model using the sample prediction result and the sample labeling result to obtain a binding affinity detection model to detect a binding affinity between a target protein and a target small drug molecule.

14. The apparatus according to claim 13, wherein the determining code is further configured to cause at least one of the at least one processor to:
generate structure data of a plurality of candidate complex conformations based on the protein structure data and the small molecule structure data;
call the neural network model to determine quality indicators of the plurality of candidate complex conformations based on the structure data of the plurality of candidate complex conformations; and
call the neural network model to select the plurality of target complex conformations from the plurality of candidate complex conformations based on the quality indicators to obtain the structure data of the plurality of target complex conformations.

15. The apparatus according to claim 14, wherein the protein structure data comprises at least one type; and
wherein the determining code is further configured to cause at least one of the at least one processor to:
for the at least one type of protein structure data, generate structure data of a candidate complex conformation corresponding to the type of protein structure data based on the type of protein structure data and the small molecule structure data; and
determine the structure data of the plurality of candidate complex conformations based on structure data of candidate complex conformations corresponding to various types of protein structure data.

16. The apparatus according to claim 14, wherein the determining code is further configured to cause at least one of the at least one processor to:
determine graph structures of the plurality of candidate complex conformations based on the structure data of the plurality of candidate complex conformations, wherein the graph structure of any candidate complex conformation represents a spatial structure of the candidate complex conformation;
call the neural network model to determine conformational features of the plurality of candidate complex conformations based on the graph structures of the plurality of candidate complex conformations; and
call the neural network model to determine the quality indicators of the plurality of candidate complex conformations based on the conformational features of the plurality of candidate complex conformations.

17. The apparatus according to claim 16, wherein the structure data of any of the plurality of candidate complex conformations comprises atomic data of a plurality of atoms, and any one of the atoms is an atom in the sample protein or an atom in the sample small drug molecule;
wherein the graph structure of the candidate complex conformation comprises a plurality of nodes and a plurality of edges; and
wherein the determining code is further configured to cause at least one of the at least one processor to:
for each candidate complex conformation, determine distance data between every two atoms based on the atomic data of the plurality of atoms comprised in each candidate complex conformation;
determine the nodes comprised in the graph structure of each candidate complex conformation based on the atomic data of the atoms comprised in each candidate complex conformation; and
for any two nodes comprised in the graph structure of each candidate complex conformation, based on the distance data between two atoms corresponding to the two nodes being less than a distance threshold, add an edge between the two nodes.

18. The apparatus according to claim 13, wherein the determining code is further configured to cause at least one of the at least one processor to:
call the neural network model to determine weights of the plurality of target complex conformations based on the conformational features of the plurality of target complex conformations, the conformational feature of each target complex conformation being determined based on the structure data of each target complex conformation; and
call the neural network model to determine the sample prediction result based on the conformational features and the weights of the plurality of target complex conformations.

19. The apparatus according to claim 18, wherein the determining code is further configured to cause at least one of the at least one processor to:
call the neural network model to determine weighted conformational features of the plurality of target complex conformations based on a reference weight and the conformational features of the plurality of target complex conformations; and
call the neural network model to perform normalization processing on the weighted conformational features of the plurality of target complex conformations to obtain the weights of the plurality of target complex conformations.

20. A non-transitory computer-readable storage medium storing computer code which, when executed by at least one processor, causes the at least one processor to at least:
obtain protein structure data of a sample protein, small molecule structure data of a sample small drug molecule, and a sample labeling result, the sample labeling result being obtained through labeling and indicating a binding affinity between the sample protein and the sample small drug molecule;
call a neural network model to determine structure data of a plurality of target complex conformations based on the protein structure data and the small molecule structure data;
call the neural network model to determine a sample prediction result based on the structure data of the target complex conformations; and
train the neural network model using the sample prediction result and the sample labeling result to obtain a binding affinity detection model configured to detect a binding affinity between a target protein and a target small drug molecule.
